(19) United States
(12) Patent Application Publication
(10) Pub. No.: US 2022/0096600 A1
(43) Pub. Date: Mar. 31, 2022

(54) PERIOSTEAL SKELETAL STEM CELLS IN BONE REPAIR

(71) Applicant: Baylor College of Medicine, Houston, TX (US)

(72) Inventors: Dongsu Park, Houston, TX (US); Laura Ortinau, Manvel, TX (US)

(21) Appl. No.: 17/310,119

(22) PCT Filed: Feb. 7, 2020
(86) PCT No.: PCT/US2020/017301

§ 371 Date: Jul. 16, 2021

Related U.S. Application Data
(60) Provisional application No. 62/802,616, filed on Feb. 7, 2019.

(51) Int. Cl. A61K 38/19; A61P 19/08; A61K 38/18; C07K 14/52; A61L 27/36; C07K 16/24; C07K 14/51

(52) U.S. Cl. CPC A61K 38/19; A61P 19/08; A61K 38/18; C07K 14/51; A61L 27/36; C07K 16/24; C07K 14/52

(57) ABSTRACT

Embodiments of the disclosure encompass methods and compositions for bone repair and bone injury healing. In some embodiments, the bone repair and bone injury healing utilizes the enhancement of migration of certain types of bone cells upon stimulation by a particular cytokine. In specific embodiments, migration of periosteal skeletal stem cells upon delivery of CCL5 and/or TNFα is enhanced and fosters bone repair and healing.

This application claims priority to U.S. Provisional Patent Application Ser. No. 62/802,616, filed Feb. 7, 2019, which is incorporated by reference herein in its entirety.

This invention was made with government support under 1 KO1 AR061434 and 1R01 AR072018 both awarded by National Institutes of Health. The government has certain rights in the invention.

### TECHNICAL FIELD

Embodiments of the disclosure include at least the fields of molecular biology, cell biology, genetics, and medicine.

### BACKGROUND

Stem cells are indispensable for the repair of most tissue injuries through distinct regulatory mechanisms (Xin et al., 2016), and dysregulation of these cells often leads to impaired/delayed healing (Riminucci et al., 2015). In particular, multipotent skeletal stem cells (SSCs) are known to exist within the bone marrow (BM) and provide mature osteoblasts, chondrocytes, adipocytes, and marrow stromal cells required for skeletal development, homeostasis, and repair (Bianco and Robey, 2015). More recently, SSC populations have been identified within tissues other than BM (e.g. calvarial sutures and the periosteum), indicating that endogenous SSCs have multiple tissue locations. Yet, which SSCs recognize tissue injury and how they initiate the repair process remains a fundamental question.

The existence of diverse stem cell pools characterized by distinct molecular markers and context-dependent functions has been reported in multiple tissues (Goodell et al., 2015). Tissue-resident SSCs are also known to be a heterogeneous population with different locations. Long-term repopulating SSCs that contribute to bone regeneration and repair have been identified within the BM (Caplan, 1991; Friedenstein et al., 1974; Morikawa et al., 2009) (Chan et al., 2009; Chan et al., 2015), and they are a subset of BM perivascular cells marked by Cxcl12-DTR-GFP (Omatsu et al., 2010), Nestin-GFP (Mendez-Ferrer et al., 2010), Prx1-Cre (Greenbaum et al., 2013), LepR-Cre (Zhou et al., 2014), or Mx1-Cre (Park et al., 2012a). Interestingly, a non-perivascular SSC population with in vivo osteogenic and chondrogenic potential that is distinctly different from perivascular SSCs is labeled by Gremlin 1 (Grem1) (Worthley et al., 2015). Similar to BM, osteo/chondrogenic stem/progenitors have also been found in the periosteum and are reported to have an important role in fracture repair (Roberts et al., 2015). These Prx1- or Axin2-expressing periosteal progenitors contribute to outer bone shaping, cortical thickness, and fracture repair (Ouyang et al., 2013; Ransom et al., 2016; Roberts et al., 2015; Wilk et al., 2017). In addition, these periosteal cells are particularly important in the life-long repair of bones with limited or no BM, suggesting that SSCs reside in the periosteum.

Fracture repair is a dynamic multi-cellular process distinct from embryonic skeleton development and remodeling. During this process, the rapid recruitment, proliferation, and subsequent differentiation of skeletal stem/progenitor cells are critical for overall stability and fixation of fractures (Einhorn and Gerstenfeld, 2015). Multiple cytokines and growth factors such as Wnt, FGF2, PDGF, and BMPs are known to regulate bone remodeling and repair and have been tested for the enhancement of skeletal repair in critical segment defects (Einhorn and Gerstenfeld, 2015; Schindeler et al., 2008). However, because of the lack of specific markers to distinguish SSCs at different locations and the cellular complexity of injury models generated through disruption of the bone compartment's physical barrier, the direct effect of these molecules on endogenous SSCs during injury healing is unclear. New animal models have been developed that allow real-time tracking of the recruitment and activation of endogenous SSCs at different locations, and sought to identify the origin and unique characteristics of periosteum-resident SSCs (P-SSCs) responsible for cortical bone healing. However, the ability to specifically recruit such cells to a specific site is needed in the art, as is the need for bone repair treatments.

### BRIEF SUMMARY

The present disclosure is directed to systems, methods, and compositions for administering to a specific site in an individual in need. In specific embodiments, the site is one or more bone sites. The site in need may be a bone break, bone fracture, bone degeneration, site of bone irradiation, site of osteoporosis, site of osteopetrosis, site of osteosclerosis, or other bone degenerative diseases, or a site that would benefit from prophylactic systems, methods, or compositions to delay or inhibit bone degeneration. The individual may also be administered one or more other conventional or novel methods and/or compositions to aid in the bone healing process. Such methods include, for example, surgery, placing the individual in a cast or sling, a fixation device, or a combination thereof. The site in need may be monitored for severity of need and also may be monitored for efficacy of the disclosed systems, methods, and/or compositions during and/or after the administration. One skilled in the art can determine the efficacy of the disclosed systems, methods, or compositions and adjust the dosage and timing of the administration in order to effectively induce bone healing in the individual.

In some embodiments, there are methods of inducing bone healing in an individual comprising administering one or more cytokines to the individual at a site in need, including at a bone site. In particular embodiments, the disclosure is directed to methods of inducing bone healing at least at one site in need in an individual. In specific embodiments, the disclosure is directed to methods of recruiting periosteal skeletal stem cells (P-SSCs) to the site in need. The P-SSCs may differentiate into osteoblasts and aid in the induction of bone healing, in at least some cases. In specific embodiments, the disclosure is directed to methods to reduce osteoclast migration to a site in need, wherein osteoclasts may reduce the induction of bone healing. Examples of the embodiments provided may be achieved through the administration of at least one cytokine at the site in need of bone healing. The cytokine administered may be, in particular embodiments, CCL5 and/or TNFα. In some embodiments, stem cells, including for example P-SSCs, may be administered to an individual. The administered P-SSCs may express Mx1 and/or αSMA. The P-SSCs may be administered locally (such as at a site in need) and/or systemically.

In particular embodiments, the disclosure is directed to methods of impairment of bone development at a specific site in an individual, for example wherein the recruitment of osteoclasts is desired. The method of impairing bone development may be achieved through the administration of one or more compositions that inhibit the function of CCR5, TNFα, or both. An individual may be in need of a reduction of bone healing or development, such as with one or more bone spurs or one or more sites of ectopic bone formation or one or more bunions, for example.

Administration of any composition encompassed by the disclosure may comprise injection or delivery of the cytokine(s), stem cells, or other composition(s) into the individual at the site in need. The cytokine or composition may be administered with a biocompatible gel or scaffold, which may allow for local cytokine signaling. The cytokine or composition may be formulated with a biocompatible gel or scaffold prior to administration, wherein the formulation is a new composition. The cytokine(s), cells, or composition(s) can be administered a single time or the cytokine(s), cells, or composition(s) can be administered multiple times, wherein the number of administrations needed is assessed by one skilled in the art.

Embodiments of the disclosure include methods of inducing bone healing in an individual comprising administering at least one cytokine to the individual at a site in need, wherein the cytokine is selected from the group consisting of CCL5, TNFα, BMP2, Wnt, or a combination thereof. In some cases, administering of the cytokine comprises delivery directly to the site in need (as an example, by injection), although in some cases it is not, such as being systemic. Administering may or may not comprise delivery of the cytokine with a gel or scaffold (for example, matrigel, hydrogel, periosteum, hydroxyapatite, tricalcium phosphate, polystyrene, poly-1-lactic acid, polyglycolic acid, poly-dl-lactic-co-glycolic acid, collagen, proteoglycans, alginate-based substrates, chitosan, collagen-GAG, extracellular matrix, or a combination thereof).

Administration of any composition of the disclosure may or may not comprise delivery of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng of cytokine to the site in need. Administration of any composition of the disclosure may or may not comprise delivery of at least about 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000, or more stem cells. The administering of any composition may or may not comprise at least one delivery.

In particular cases, the delivery indirectly or directly provides the composition to a site in need, such as a bone fracture, bone break, bone degeneration, bone irradiation, a site of osteoporosis, or a combination thereof. The site in need may or may not be monitored for bone regeneration.

In specific embodiments, the administering comprises administration of at least one cytokine and at least one additional therapy for the site in need, such as surgical repair, fixation of a device to set the site in need, placing the individual in a cast or sling, or a combination thereof.

Embodiments of the disclosure include methods to recruit periosteal skeletal stem cells to a site in an individual in need thereof, comprising administering at least one cytokine to the individual at a site in need, wherein the cytokine is selected from the group consisting of CCL5, TNFα, BMP2, Wnt, or a combination thereof.

Any individual treated with methods provided herein may or may not have osteopetrosis or osteosclerosis.

Particular embodiments encompass methods to reduce osteoclast migration to a site in need in an individual comprising administering at least one cytokine to the individual at a site in need, wherein the cytokine is selected from the group consisting of CCL5, TNFα, BMP2, Wnt, or a combination thereof.

Embodiments of the disclosure include methods for inhibiting bone formation at a site in need in an individual comprising administering at least one agent that inhibits the function of CCR5 TNFα, BMP2, or Wnt. The agent may or may not be maraviroc or an antibody. One example of bone formation to be inhibited is a bone spur or bunion. Administration of the agent may or may not comprise delivery directly to the site in need and may comprise delivery of the agent with a gel or scaffold. Administration may or may not comprise delivery of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM of agent to the site in need. Administration may comprise at least one delivery to the individual. In at least some cases, the site in need comprises a bone spur or ectopic bone formation site or bunion. The site in need may be monitored for the presence or absence of bone regeneration, or bone loss. In some embodiments, the administration to the individual may be prophylactic.

Embodiments of the disclosure include compositions for administering to a bone site comprising one or more cytokines and/or agents, and optionally one or more gels and/or scaffolds. In specific cases, the cytokine and/or agent comprises CCL5, TNFα, BMP2, Wnt, maraviroc, an antibody that targets CCL5, an antibody that targets TNFα, an antibody that targets BMP2, an antibody that targets Wnt, an antibody that targets CCR5 or a combination thereof. The gel and/or scaffold may or may not comprise matrigel, hydrogel, periosteum, hydroxyapatite, tricalcium phosphate, polystyrene, poly-1-lactic acid, polyglycolic acid, poly-dl-lactic-co-glycolic acid, collagen, proteoglycans, alginate-based substrates, chitosan, collagen-GAG, extracellular matrix, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1G show Mx1⁺αSMA⁺ periosteal cells highly express SSPC markers and contribute to bone injury repair. Mx1-Cre activity was induced at 4-weeks of age in Mx1/Tomato/αSMA-GFP mice with 5 doses (10 days) of pIpC. Two-month old Mx1/Tomato/αSMA-GFP dual reporter mice were lethally irradiated and received wild type-bone marrow transplant (10⁶ cells) to reduce the background of hematopoietic lineage cells. FIGS. 1A & 1B. The periosteal Mx1⁺αSMA⁺ population in the tibia and calvaria suture of Mx1/Tomato/SMA-GFP dual reporter mice was analyzed by immunofluorescence staining (FIG. 1A) and intravital microscopy (FIG. 1B), respectively. Arrows indicate Mx1⁺αSMA⁺ cells (red and green) in the periosteum (P, FIG. 1A) and suture (FIG. 1B), Mx1⁺αSMA⁻ osteoblasts (Mx1⁺ Obs, red) in the endosteum, and αSMA⁺ cells (SMA+, green) in muscle. M: muscle; B: bone; BM: bone marrow. FIG. 1C. High expression of SSPC surface markers and transcription factors in the Mx1⁺αSMA⁺ fraction in the periosteum. Graph shows percentages of SSPC surface markers (CD105⁺ and CD140a⁺) and relative expression of SSPC transcription factors (Runx2, Cxcl12, LepR, & Gremlin) from periosteal Mx1⁺αSMA⁺ cells in comparison to those of Mx1⁺αSMA⁻, Mx1⁻αSMA⁺, Mx1⁻αSMA⁻, or CD45⁺ cells from the periosteum determined by flow cytometry (n=6). 1D. A representative single-cell-driven hemisphere colony formation of Mx1⁺αSMA⁺ periosteal cells and their osteogenic, adipogenic, and chondrogenic potential. Alizalin red, oil red O, and Toluidine-blue staining were used for osteogenic, adipogenic, chondrogenic differentiation, respectively. 1E. Injury-mediated expansion of the Mx1⁺ and Mx1⁺αSMA⁺ populations (red and white arrows) in the callus and new periosteum (New PO, red arrow), but not in bone marrow (BM), supply the majority of the new bone-forming osteoblasts (new bone) two weeks post-injury (representative of 5 independent experiments). Mx1⁺αSMA⁺ (red and green) cells and their contribution to new chondrocytes (left, arrows) and osteoblasts (right, arrows) were assessed by immunostaining of the injury sites with anti-aggrecan antibody (left, white) and anti-osteocalcin antibody (right, white). 1F. After calvaria injuries in Mx1/tomato/αSMA-GFP dual reporter mice at 4 months of age, Mx1⁺αSMA⁺ (yellow) periosteal cells (DO injury) and their repopulation at injury sites (D21 injury) were assessed by sequential in vivo imaging. Ob, osteoblast. 1G. 3-D reconstruction of in vivo z-stack images at twenty-one days post-injury of the calvaria show that Mx1⁺αSMA⁺ (red and green) cells remain undifferentiated in periosteum (left) and in the middle of injury (right, yellow), while they become Mx1⁺αSMA⁻ osteoblasts (layer a, arrow) near bone surface and/or osteocytes (layer b, arrow) in new bone (blue).

FIGS. 2A-2F shows Mx1⁺ osteogenic progenitors in the periosteum supply the majority of osteoblasts in cortical bone injury. Four-week-old Mx1/Tomato/Ocn-GFP dual reporter mice were lethally irradiated and received wild type-bone marrow transplant (10⁶ cells) to reduce the background of hematopoietic lineage cells. Mx1-Cre activity was induced at 2-months of age in Mx1/Tomato/Ocn-GFP mice with 5 doses (10 days) of pIpC. FIGS. 2A-2C. Periosteal Mx1⁺ progenitor cells (Mx1+: red, arrows) and endosteal osteoblasts (green) and new osteoblasts from Mx1⁺ progenitor cells (Mx1+Ocn+: red and green) in the tibial metaphysis and diaphysis of Mx1/Tomato/Ocn-GFP mice were analyzed by immunofluorescent staining (FIGS. 2A and 2B) and intravital imaging (FIG. 2C). Graph in A shows percentage of Mx1⁺ (Tomato+) progenitor cells in the periosteum of metaphysis and diaphysis (n=5). PO: periosteum; M: muscle; B: bone; BM: bone marrow; FL: fibrous layer; CL: Cambium layer. FIG. 2D. Mx1⁺ progenitors in the periosteum show SSC characteristics. Hematopoietic cells (CD45⁺) and non-hematopoietic cells (CD45⁻CD31⁻Ter119⁻): bone marrow stromal cells (CD105⁻CD140⁺), osteoblasts (Ocn⁺), Mx1⁻ (CD105⁺CD140⁺Mx1⁻), and Mx1⁺ (CD105⁺CD140⁺Mx1⁺) SSCs from Mx1/Tomato/Ocn-GFP mouse bones were sorted, and the levels of the indicated genes were quantified by qPCR. ** P<0.01; ***P<0.001. FIGS. 2E & 2F. Representative tibia drill-hole defect (FIG. 2E) or periosteal defect (FIG. 2F, defect on cortical bone surface) showing that Mx1⁺ periosteal progenitors (red) contribute the majority of callus-forming osteoblasts (Mx1+Ocn+) in the injury callus (left box in FIG. 2F) 2 weeks after injury. Expansion of Mx1⁺ progenitors (red) in new periosteum (PO) at the injury site is indicated by the arrows (arrow in FIG. 2E middle and white box in FIG. 2F top). Graph shows percentage of Mx1⁺Ocn⁺ (Tomato+ and GFP+) osteoblasts from uninjured endosteum (normal) and injured bone callus (Callus) (n=5).

FIGS. 3A-3F show Mx1⁺ P-SSCs are necessary for bone healing and remain in the periosteum after injury. FIG. 3A. The periosteum of Mx1/tomato/iDTR mice were treated locally with a low-dose of ditheria toxin (+DT; 20 μL at 1 μg/mL) or control (−DT; PBS) for seven days to ablate Mx1⁺ (tomato⁺) periosteal cells, but not bone marrow cells. FIG. 3B. In vivo imaging before injury, the DT-mediated reduction of the Mx1⁺ (red) populations in the periosteum (PO) without change of Mx1⁺ bone marrow (BM) resident osteoblasts (BM obs) or osteocytes within the bone (B, blue) was analyzed. FIG. 3C. After injury, the numbers of Mx1⁺ (red) cells at the calvarial injury sites were tracked at the indicated time points using in vivo imaging of the bone injury. Local ablation of Mx1⁺ periosteal cells prevents osteoprogenitor/osteoblast expansion at the injury sites, resulting in a significant reduction in bone injury repair. FIG. 3D. Calvarial bone healing of Mx1/tomato/iDTR mice treated with DT (+DT) or PBS (−DT) was analyzed by μCT (bone volume/total volume; BV/TV) at day 16. Data represent at least three independent experiments. Scale bars are 100 μm. FIG. 3E. After calvaria injuries in Mx1/tomato/SMA-GFP dual reporter mice at 4 months of age (3 months after pIpC) (FIG. 1F), long-term maintenance of Mx1⁺αSMA⁺ P-SSCs (Tomato⁺GFP⁺ yellow, Pre-injury) and their repopulation at injury sites (D14 injury) were assessed by sequential in vivo imaging of a second round of injuries at 12 months of age (8 months after the first injury). Bone (blue) (n=6). FIG. 3F. High expression SSPC markers are maintained in the Mx1⁺αSMA⁺ fraction in the periosteum after a second injury. Histograms show percentages of the CD200⁺, CD140a⁺, and Vcam-1⁺ SSC marker expression within Mx1⁺αSMA⁺ P-SSCs (red) in comparison to those of SMA-GFP⁺ cells (gray) from the periosteum determined by flow cytometry (n=2).

FIGS. 4A-4G show rapidly migrating Mx1⁺αSMA⁺ P-SSCs uniquely express CCR5 in vivo. FIG. 4A. Using Mx1/Tomato/SMA-GFP dual reporter mice (3-month old, 2 months post pIpC from FIG. 1A), the early injury response of Mx1⁺αSMA⁺ periosteal cells (Periosteum) and suture mesenchymal cells (Suture) near the injury site was imaged at the indicated times after injury (>5 replicates). Arrows indicate migrating Mx1⁺αSMA⁺ P-SSCs. Bone (blue), αSMA⁺ cells (green), Mx1⁺ periosteal cells (red), Mx1⁺αSMA⁺ P-SSCs (red and green). Scale bar, 100 μm. FIG. 4B. List of cell migration genes that are most abundantly expressed in Mx1⁺ periosteal cells (Mx1+PCs) compared to control populations (FIG. 9D). FIG. 4C. Chemotaxis gene set analysis of Mx1⁺ periosteal cells (Mx1+PCs) vs. osteocalcin-GFP⁺ osteoblasts (Ocn+obs) (FIG. 9D). FIG. 4D. Relative cell surface expression of CCR5 (top histograms, graph) and CXCR4 (bottom histograms) in Mx1⁺αSMA⁺ P-SSCs (M+S+) from 3-4-month-old Mx1/Tomato/SMA-GFP dual reporter mice, compared to those of indicated cells and CD45+ cells, was analyzed by flow cytometry. FIG. 4E. Relative cell surface expression of CCR5 in CD45⁺, Mx1⁺CD140a⁺, and αSMA⁺ CD140a⁺ bone marrow cells. FIG. 4F. Relative cell surface expression of CCR5 and CD140a in control CD45±CD31+(45/31+) cells, 45⁻31⁻GFP⁻ stroma cells, or Prx1CreER-GFP⁺ cells from the periosteum of 1-month-old Prx1CreER-GFP⁺ mice. FIG. 4G. In vivo imaging of periosteal Prx1⁺ (green) population in the calvaria of Prx1CreER-GFP reporter mice without tamoxifen induction (top image). Mx1/tomato/Prx1-GFP mice were treated with 5 doses of pIpC every other day to induce Mx1 labeling (red); followed by lethal irradiation and transplantation of wild-type BM. In vivo imaging of periosteal Prx1+(green) and Mx1+(red) populations in the calvaria of Mx1/tomato/Prx1-GFP reporter mice without tamoxifen induction (bottom images). Box represents Prx1⁺Mx1⁺ (red and green) cells.

FIGS. 5A-5D shows the migration of P-SSC is induced by CCL5 in vitro and in vivo. FIG. 5A. The effects of TNF-α (10 ng/mL), CCL5 (20 ng/mL), PDGF-β (20 ng/mL), BMP (20 ng/mL), and TGF-β (10 ng/mL) on FACS-sorted Mx1⁺ P-SSCs (5,000/well) migration were examined, using a Transwell Assay (40-μm pore), for 12 hours, followed by their colony formation for 4 days. Relative migrating cell numbers from three independent experiments are shown on the right. *P<0.05; **P<0.01. FIGS. 5B & 5C. Mx1/Tomato/SMA-GFP mice were treated with 5 doses (10 days) of pIpC to induce Mx1 labeling (red); followed by lethal irradiation and transplantation of wild-type BM. Analysis were performed at least 1 month after WTBMT. FIG. 5B. The average in vivo migration distance of individual cells induced by TNFα, CXCL12, or CCL5 was measured in Mx1/Tomato/SMA-GFP mice. FIG. 5C. In vivo real-time imaging shows that CCL5, but not TNFα or CXCL12 (all 2 μL at 10 ng/μL of Matrigel), at the injury site induced the migration of Mx1⁺αSMA⁺ P-SSCs (>3 replicates). The numbers indicate the time of imaging. Dotted line indicates the migratory path of Mx1⁺αSMA⁺ (yellow) cells. Scale bar for TNFα and CXCL12 treatment is 50 μm and CCL5 treatment is 20 μm. FIG. 5D. CCL5 induced in vivo real-time migration of Prx1+(green) P-SSCs as assessed in Prx1CreER-GFP reporter mice treated with CCL5 (2 μL at 10 ng/μL of Matrigel) or Matrigel alone (2 μL, Control). Dotted line indicates migratory path of Prx1⁺ (GFP⁺) P-SSCs. Bottom images represent sequential imaging of Prx1⁺ P-SSC migration over 16 minutes (1 image per minute). Scale bar is 50 μm.

FIGS. 6A-6F show CCL5 treatment following bone injury induces P-SSC recruitment and activation leading to accelerated bone healing. FIG. 6A. Representative μCT images of proximal tibial injuries (left) of age- and sex-matched WT (n=12) or 2- to 4-monthold Ccl5^{-/-} mice 10 days post injury (n=13). BV/TV of external callus (left) and injury sites (right) was assessed. FIG. 6B. WT-BL6 mice (n=10 per group) were irradiated (9.5 Gy) and transplanted with 106 WT (WT-BMT) or Ccl5^{-/-} BM mononuclear cells (Ccl5^{-/-} BMT). Five weeks later, BV/TV of injury sites was assessed at 10 days after tibial drill-hole injuries. FIG. 6C. Sequential imaging of 3-4-month-old Mx1/Tomato/αSMA-GFP dual reporter mice prepared from FIG. 1 and treated with supplemental CCL5 (2 μL at 10 ng/μL of Matrigel), CXCL12 (2 μL at 10 ng/μL of Matrigel), or or Matrigel alone (2 μL, con) at the indicated times post-injury. White dotted line indicates original injury sight; white bar is 100 μm. FIG. 6D. Representative μCT images of calvaria injuries (from 6A) treated with CCL5, or CXCL12, ten days post-injury (left). Bone volume/total volume (BV/TV) was assessed for the quantification of bone healing (right). FIG. 6E. Representative μCT images of proximal tibia injuries (left) treated with Matrigel (2 μL Matrigel; CON) or CCL5 (2 μL at 10 ng/μL of Matrigel; +CCL5) on Day 0, 2, and 4 post-injury The CCR5 inhibitor was injected (10 μL at 2.5 μg/μL in Matrigel) locally for 7 days post-injury. BV/TV was used to determine the effects of CCL5 or CCR5 inhibitor treatment on bone healing (right). FIG. 6F. Representative μCT images of tibia injury treated locally with Matrigel (2 μL Matrigel; Control) or a CCR5 inhibitor (10 μL at 2.5 μg/μL in Matrigel; +R5 Inhibitor) on day 0, 2, 4 post injury. On Day 7 post-injury, subsequent bone volume/total volume (BV/TV) analysis was used for the quantification of bone healing (right). FIG. 6G. Single-cell driven colonies from human periosteal tissue were cultured for 7-10 days (P1), then analyzed for expression of CCR5 and SSC markers (CD105 and CD140a). Isotype controls were used as negative controls (blue).

FIGS. 7A-7F show CCR5-CCL5 influences periosteal cell migration and bone healing. FIG. 7A. The external callus volume (left), BV/TV (right), and mCT images of proximal tibial bone injuries in WTor Ccr5^{-/-} mice at 10 days post injury were used to assess bone healing. FIG. 7B. Sequential in vivo imaging of Mx1/Tomato/αSMA-GFP mouse (3-month-old) calvarial injuries treated with a CCR5 inhibitor (+R5 Inhibitor; 10 mL at 2.5 mg/mL Maraviroc in Matrigel) or gel control (10 mL of Matrigel) for 10 days post injury. FIG. 7C. Representative mCT images on day 7 post injury (FIG. 7C, left) and BV/TV analysis (FIG. 7C, right graph) were used for the quantification of bone healing. FIG. 7D. Primary cells isolated from human periosteal tissue (Human PO) were analyzed for expression of CCR5 and indicated SSC markers. Dotted line, isotype controls. 7 FIG. E. CCL5 induces human periosteal cell migration. The effects of CCL5 (20 ng/mL) or TNFα (20 ng/mL) on the migration of human periosteal cells (10,000/well) were determined by in vitro scratch assay and counting the migrating cells within 6 hours. FIG. 7F. A transwell migration assay (8-μm pore) was used to determine the effects of CCL5 (5 ng/mL) or Cxcl12 (5 ng/mL) on human periosteal migration (P1, 15,000/well) migration for 24 h.

FIGS. 8A-8E show Mx1⁺αSMA⁺ P-SSCs are the source of new osteoblasts in fracture healing and remain in the periosteum after injury, related to FIGS. 1A & 1B. The periosteal Mx1⁺αSMA⁺ population in the tibia (FIG. 8A) and calvaria suture (FIG. 8B) of Mx1/Tomato/SMA-GFP dual reporter mice was analyzed by immunofluorescence staining. Arrows indicate Mx1⁺αSMA⁺ cells (red and green) in the periosteum (FIG. 8A) and suture (FIG. 8B), Mx1⁺αSMA⁻ osteoblasts (Mx1⁺Obs, red) in the endosteum, and αSMA⁺ cells (SMA+, green) in muscle. M: muscle; B: bone; BM: bone marrow. FIG. 8C. High expression of CD105 and CD140a SSPC markers in the Mx1⁺αSMA⁺ fraction in the periosteum. Graph shows percentages of the CD105⁺CD140a⁺ SSC population within Mx1⁺αSMA⁺, Mx1⁻αSMA⁺, or Mx1⁺αSMA⁻ cells from the periosteum determined by flow cytometry (n=6). FIG. 8D. Fourteen days after transverse tibial fracture, robust contribution of the Mx1⁺ (Tomato⁺) periosteal cells to the external callus (box a), repopulation of Mx1⁺αSMA⁺ (Tomato⁺GFP⁺) cells in the new periosteum (white arrow, New PO), and their contribution to cartilaginous callus (box b) were assessed by immunostaining with anti-GFP antibody. Intermedullary pin insertion (*), fractured bone (dot lines) and BM are indicated (tiled image, n=5). FIG. 8E. 3-D reconstruction of in vivo z-stack images of injury shown in FIG. 1F. Mx1⁺ (tomato) and αSMA⁺ (GFP) cells newly appear at the injury sight by day 7. By day 21 the injury is filled with more differentiated Mx1⁺αSMA⁻ cells while Mx1⁺αSMA⁺ P-SSCs mainly reside in the outer surface of the injury.

FIGS. 9A-9C show Mx1⁺ periosteal cells are clonogenic progenitors responsible for callus-forming cells in vivo, related to FIG. 2. FIG. 9A. Schematic representation of Mx1-Cre; Rosa26-Tomato; osteocalcin-GFP mice (left) and a tibia section of Mx1/tomato/Ocn-GFP mice showing distinct metaphyseal Mx1⁺ progenitors (red) and their differentiated mature osteoblasts (Mx1⁺Ocn⁺, red and green) present in bone marrow 4 weeks after pIpC treatment (+pIpC), followed by lethal irradiation and wild-type bone marrow transplantation (+WT-BMT). FIG. 9B. After induction of Mx1 with pIpC and WT-BMT, single cell cultures of FACS-sorted Mx1⁺ periosteal cells displayed colony formation (Sphere shape) and their osteogenic differentiation potential (Alizalin staining) after 2 weeks of differentiation medium culture. FIG. 9C. Robust contribution of Mx1⁺ periosteal progenitors (red) to the majority of new periosteal cells and callus-forming osteoblasts (red and green), without detectable responses of Mx1⁺ bone marrow cells (tomato) 2 weeks after proximal tibia injury.

FIGS. 10A-10E show Mx1⁺ periosteal cells distinct from Nestin⁺ BM-SSCs supply new osteoblasts in fracture healing, related to FIG. 3. FIG. 10A. Representative in vivo image of the periosteum (left) and bone marrow perivascular cells (middle) and an immunofluorescence image of tibia section (right) from Mx1/Tomato/Nestin-GFP mice. FIG. 10B. The percentages of Mx1⁺Nestin⁺, Nestin⁺, or Mx1⁺ skeletal progenitors in bone were determined by flow cytometry. FIG. 10C. Heatmaps representing the relative gene expression level of SSC markers compared with ˜10,000 public microarray datasets (global-scale meta-analysis with Gene Expression Commons). FIG. 10D. The relative expression level of SSC markers (Runx2 and Cxcl12) in the indicated cells (n=5 per group). CD45⁺ hematopoietic cells (CD45), GFP cells from Osteocalcin-GFP mice (Ocn), GFP cells from Osterix-GFP mice (Osx), and Mx1⁻Nestin⁺ (Mx−N+) and Mx1⁺Nestin⁺ (Mx+N+) cells within the CD105⁺CD140a⁺ population from Mx1/Tomato/Nestin-GFP mice and Mx1⁺Ocn⁻ periosteal cells (Mx1+PCs) within the CD105⁺CD140a⁺ population from Mx1/Tomato/Ocn-GFP mice were used. * P<0.05. FIG. 10E. Mx1⁺ SSCs (Red), but not Nestin-GFP+ cells (Green), supply the majority of osteoblasts in fracture healing. Mx1⁺ (Tomato) and Nestin-GFP+ cells near the injury site on calvaria of Mx1/Tomato/Nestin-GFP mice were imaged at the indicated times after injury.

FIGS. 11A-11D show LepR⁺ periosteal cells are distinct from LepR⁺ bone marrow cells and supply new osteoblasts in fracture healing, related to FIG. 4. FIG. 11A. In vivo imaging of LepR-Cre/Tomato reporter mice. LepR⁺ (tomato) cells are present in the periosteum, suture, and bone marrow (BM) of the calvaria. FIG. 11B. In vivo imaging of LepR⁺ (tomato) cells in response to a calvaria injury on days 0, 4, 7, and 12 post-injury. Arrows indicate migrating and proliferating LepR⁺ periosteal cells near the injury site. FIG. 11C. LepR⁺ cell proliferation occurs on the periosteum in response to injury, but not within the bone marrow. FIG. 11D. CCR5 and CD140a expression of LepR⁺ cells isolated from the periosteum or bone marrow was analyzed by flow cytometry.

FIG. 12 shows an example of a 1.1 mm tibia drill-hole defect was created (left) and injury repair was assessed 10 days later. Representative μCT images of proximal tibia injuries (Middle) of age and sex-matched WT or 2-4-month-old CCL5^{-/-} mice ten days post-injury. Bone volume/total volume (BV/TV) was used to quantify bone healing (right).

FIG. 13 shows tibia sections of Mx1/Tomato/Ocn-GFP mice two weeks after pIpC treatment demonstrating that most periosteal Mx1+ cells are undifferentiated (Mx1⁺Ocn⁻) and reside in the inner cambium layer (CL), rather than the fibrous layer (FL). Differentiated osteoblasts (Mx1⁺Ocn⁺, red and green) in the endosteal surface are indicated by green arrows (tiled image, n=5). C. Increase in Mx1□ progenitor contribution to the periosteal osteoblasts and osteocytes (right bottom) over time. 16 weeks after pIpC induction, newly differentiated osteoblasts (Mx1⁺Ocn⁺: red and green) and osteocytes (Mx1⁺Ocy) from Mx1⁺ periosteal progenitor cells in the periosteum and endosteum of Mx1/Tomato/Ocn-GFP mice (˜6-month old, 16 weeks post-pIpC) were analyzed by anti-GFP immunofluorescent staining (n=3).

FIG. 14 shows serial transplantation of Mx1⁺αSMA⁺ P-SSCs. Four weeks after transplantation of sorted Mx1⁺αSMA⁺ P-SSCs onto calvarial injury sites of C57BL/6 mice (5,000 cells/mouse), repopulating Mx1⁺αSMA⁺ P-SSCs in the periosteum (PO, top left, tiled images, Tomato⁺GFP⁺) and their osteogenic differentiation (topmiddle, white arrow) in newly generated bones (blue, middle dotted line) were analyzed by in vivo imaging. After re-sorting and transplantation of Mx1⁺αSMA⁺ P-SSCs (top right) into secondary injuries of C57BL/6 mice (˜500 cells/mouse), their periosteal repopulation (PO, bottom left), osteogenic differentiation (bottom middle, white arrow) in new bones (blue), and SSC marker expression (CD105 and CD140a, bottom right) were analyzed by in vivo imaging and flow cytometry. As controls, transplantation of CD105⁺CD140a⁺Mx1⁺αSMA⁻ or CD140a⁺Mx1⁻αSMA⁺ periosteal cells was also performed (Figure S3). n=5 mice per group.

FIGS. 15A-15B show periosteal cell transplantation related to FIG. 3C. Mx1/Tomato/αSMA-GFP dual reporter mice were treated with 5 doses of pIpC every other day to induce Mx1 labeling (red) at 4 weeks of age, followed by lethal irradiation and WT-BMT. Periosteal cells from 10-12-week old Mx1/Tomato/αSMA-GFP mice were FACs-sorted for CD45-CD31-Ter119-CD105⁺CD140⁺Mx1⁺αSMA− (Mx1+αSMA−) and CD45-CD31-Ter119-CD105⁺CD140⁺Mx1-αSMA⁺ (Mx1-αSMA⁺) cell populations. Approximately 5,000 Mx1−αSMA⁺ (FIG. 15A) or Mx1⁺αSMA− (FIG. 15B) cells were transplanted onto calvarial injury sites of wild-type C57BL/6 mice. Transplanted cells were then tracked using intravital microscopy 2, 4, and 8 weeks post transplantation. Four weeks after transplantation, transplanted cells were FACs-analyzed for SSC markers (CD105 and CD140a). Data represent five independent transplants per group with comparable results.

FIGS. 16A-16C show CathepsinK (Ctsk)⁺ osteoclast fracture healing response with CCL5 treatment or CCR5 inhibition. FIG. 16A. Wildtype mice were lethally irradiated and transplanted with bone marrow from CtskCre/Tom mice (CtskCre-BM) at 7 weeks of age. Calvarial injuries were induced in CtskCre-BM transplanted mice 14 weeks after transplantation, which allowed adequate time for bone marrow repopulation. Mice received one of 3 treatments at the injury sight: 1) control (2 uL Matrigel) Day 0, 2, and 4; 2) CCL5 (2 uL, 10 ng/uL in Matrigel) Day 0, 2, and 4; or 3) a CCR5 inhibitor (10 uL, 4.9 mM Maraviroc in Matrigel) on Day 0, 2, 4, 7, and 10 post-injury. Ctsk⁺ osteoclasts were tracked by in vivo imaging at the indicated time points. FIG. 16B. Intravital microscopy was performed on the day of injury as well as day 4, 7, and 10 post-injury. Bone collagen (blue), Ctsk⁺ osteoclasts (red), original injury (white dotted line), resorption expanding the injury (yellow dotted line). Scale bars are 100 μm. FIG. 16C. The average number of Ctsk⁺ osteoclasts at the injury sight were plotted from different injuries at the indicated time points. *p<0.05 control vs. CCL5 and ^{#}p<0.05 CCL5 vs. CCR5 inhibitor at specified time point.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “administer” or “administration” refers to providing a therapeutically effective amount of a composition to an individual at a site in need. The method of administration may or may not include injection, such as with a syringe, or delivery, such as implantation of the composition at the site in need. Other delivery routes are encompassed in the disclosure and detailed elsewhere herein.

When referring to “cells”, it is meant any number of cells, including a single cell, that is sufficient to induce or perform the disclosed method.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. Methods of Enhancing Bone Formation

Particular embodiments of the disclosure are directed towards methods of administration of compositions to one or more sites in need, such as a bone, in an individual. The site in need may be a bone break, bone fracture, bone degeneration, site of bone irradiation, and so forth. The individual may have an injury, osteoporosis, osteopetrosis, osteosclerosis, or other bone degenerative diseases. In some embodiments, the site may be one that would benefit from prophylactic systems, methods, or compositions to delay the onset or inhibit bone degeneration partially or fully, for example. The individual may also be administered other conventional or novel methods and/or compositions to aid in the bone healing process. Such methods include, for example, surgery, placing the individual in a cast or sling, a fixation device, or a combination thereof.

In particular embodiments, the methods are directed towards modulation of specific skeletal stem cells (SSCs), including periosteal SSCs (P-SSCs), in an individual. The P-SSCs may be positive for the Mx1 marker. These P-SSC may have the ability to differentiate into osteoblasts, which can aid in repairing a site in need. The methods disclosed herein may induce migration of the P-SSCs to a specific site by administration of one or more compositions at the specific site(s). In particular embodiments, the recruitment of P-SSCs, including those that differentiate into osteoblasts, reduces the recruitment of cells that may be, or differentiate into, osteoclasts. In some embodiments, the methods are directed towards modulation of SSCs, including osteoclast progenitors (OCPs), which may express the CathespsinK marker. OCPs may have the ability to differentiate into osteoclasts, which can reduce the healing response at the site in need.

In particular embodiments, the method is directed to induce bone healing at a site in need comprising the administration of at least one composition, such as a cytokine and/or stem cell. The site in need may be a bone break, bone fracture, bone degeneration, site of bone irradiation, osteoporosis, or other bone degenerative diseases, or a site that would benefit from prophylactic systems, methods, or compositions to delay or stop bone degeneration, for example. Bone healing may be induced by recruitment of P-SSCs to the site in need, wherein the P-SSCs contribute to bone healing, such as by differentiating into osteoblasts, for example. The cytokine administered to the site in need may be CCL5, TNFα, BMP2, Wnt, or a combination thereof. On specific combination may be CCL5 and TNFα. The stem cell administered may be a P-SSC, including an Mx1⁺αSMA⁺ P-SSC, as one example. In some cases, the stem cell is administered with the one or more cytokines. In a specific aspect, a P-SSC, such as Mx1⁺αSMA⁺ P-SSC, is administered with CCL5, TNFα, BMP2, Wnt, or a combination thereof. The P-SSC may be administered with CCL5 and/or TNFα, in some cases.

In any method of the disclosure, the one or more cytokines and/or stem cells (including P-SSCs) are both provided to an individual for the purpose of enhancing bone formation and optionally may include an additional therapy for bone formation. In such cases, the one or more cytokines, the stem cells, and/or the additional therapy are administered to the individual at the same time or in succession. In cases wherein the one or more cytokines, the stem cells, and/or the additional therapy are administered to the individual in succession, the order of delivery may be in any suitable order. In some cases, the individual is administered the one or more cytokines before, during, or after the stem cells and/or additional therapy. In some cases, the individual is administered the stem cells before, during, or after the one or more cytokines and/or additional therapy. In some cases, the individual is administered the additional therapy before, during, or after the one or more cytokines and/or stem cells.

When the individual is administered CCL5, TNFα, BMP2, Wnt, or a combination thereof, the form of the CCL5, TNFα, BMP2, Wnt, or a combination thereof may be of any kind, including in polynucleotide form, such as on a vector of any kind (viral or non-viral), or in polypeptide form. In some cases, the CCL5, TNFα, BMP2, Wnt, or a combination thereof are provided in their full form, whereas in other cases the CCL5, TNFα, BMP2, Wnt, or a combination thereof are provided in a functional fragment or derivative; in such cases, a fragment or derive is function if after delivery it is able to enhance bone formation.

### III. Methods of Inhibiting Bone Formation

In particular embodiments, an individual is in need of inhibiting bone formation, such as at a particular site. The inhibition of bone formation may be at the site of a bone spur (including heel spurs) or site of ectopic bone formation or bunion(s), for example. The bone spur may be caused by local inflammation, such as from degenerative arthritis (osteoarthritis) or tendinitis. The ectopic bone formation may be caused by surgery or trauma, such as to the hips and legs, for example from joint replacement (for example, from total hip arthroplasty) and/or a severe fracture of bone, such as the long bones of the lower leg. Such inhibition of bone formation may or may not include the induction of migration of cells that are osteoclasts or that differentiate into osteoclasts to a site in need of bone inhibition.

In other particular embodiments, the composition(s) to be administered comprises an agent that inhibits CCR5, such as maraviroc, for example. The inhibition of CCR5 may induce the migration of cells that are, or differentiate into, osteoclasts, such as OCPs, for example. The site in need in such methods may including sites of ectopic bone formation, such as in muscle or tendon tissue. Ectopic bone formation can be present at birth, be the result of genetics, or arise as a complication of certain medical conditions such as paraplegia and/or traumatic injury, as examples. The composition to be administered may comprise other materials, such as a biocompatible gel and/or scaffold, for example.

An individual in need of inhibition of bone formation may be determined to be in need of inhibition of bone formation, such as upon determination of ectopic bone formation or upon identification of a bone spur or bunion, or upon identification of a risk thereof.

### IV. Cytokines and Inhibitors

In particular embodiments, the methods disclosed are directed to administration of a composition at a site in need, such as a bone, in an individual. The composition, in some embodiments, comprises at least one cytokine. The cytokine may be CCL5 and/or TNFα. The cytokine may be produced by any known means of obtaining cytokines, including recombinant protein purification, mammalian expression constructs, viral or non-viral transfections, isolation from cellular or hematopoietic sources, or purchasing from a commercial vendor, for example. The cytokine may also be produced in the individual from a polynucleotide or viral gene therapy. The composition may be delivered in a cell, in some cases (MSCs or isolated skeletal stem cells).

In particular embodiments, the cytokine comprises the CCL5 cytokine, wherein the CCL5 cytokine is translated from the CCL5 coding sequence, including as exemplary sequences, the sequences encoded by GenBank® Accession No NM_001278736.1 (SEQ ID NO:1) or NM_002985.2 (SEQ ID NO:2), or any homologous sequence from a human or non-human species that produces the same function as the exemplary sequence provided. In cases wherein the CCL5 cytokine is employed as a polypeptide, examples of the polypeptide include GenBank Accession No. NP_001265665.1 (SEQ ID NO:3) or NP_002976.2 (SEQ ID NO:4).

In particular embodiments, the cytokine comprises the TNFα cytokine, wherein the TNFα cytokine is translated from the TNFα coding sequence, including as an exemplary sequence, the sequence encoded by GenBank® Accession No NM_000594.3 (SEQ ID NO:5), or any homologous sequence from a human or non-human species that produces the same function as the exemplary sequence provided. In cases wherein the TNFα cytokine is employed as a polypeptide, an example of the polypeptide encoded by GenBank Accession No. NM_000594.3 (SEQ ID NO:6).

In particular embodiments, the cytokine comprises the BMP2 cytokine, wherein the BMP2 cytokine is translated from the BMP2 coding sequence, including as an exemplary sequence, the sequence encoded by GenBank® Accession No NM_001200.3 (SEQ ID NO:7), or any homologous sequence from a human or non-human species that produces the same function as the exemplary sequence provided. In cases wherein the BMP2 cytokine is employed as a polypeptide, an example of the polypeptide includes GenBank Accession No. NP_001191.1 (SEQ ID NO:8).

In particular embodiments, the cytokine comprises the Wnt cytokine, wherein the Wnt cytokine is translated from the Wnt coding sequence, including as an exemplary sequence, (Wnt3a in mouse bone injury is reported to induce bone healing and regeneration; Science Translational Medicine 28 Apr. 2010: Vol. 2, Issue 29, pp. 29ra30), or any homologous sequence from a human or non-human species that produces the same function as the exemplary sequence provided.

Homologous sequences may be sequences that have silent mutations, wherein the homologous sequence produces the same amino acid sequence. Homologous sequences may also, in a non-limiting example, be sequences that have mutations that code for amino acids that are functionally or practically equivalent to the amino acid coded in the exemplary sequence. Homologous sequences may also have insertions or deletions relative to the exemplary sequence, wherein the insertions or deletions do not alter the inherent function of the translated cytokine. Homologous sequences may also be from a non-human species that encode for a protein or polypeptide that is functionally equivalent to CCL5, TNFα, BMP2, or Wnt. Methods to produce cytokines that utilize non-natural amino acids, such as to enhance the function of the cytokine or improve the stability of the cytokine, may also be used. In certain embodiments, the cytokine may comprise a biologically active portion or fragment of any of the provided full length cytokines that may be administered to the individual. The fragment or portion may comprise approximately 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the amino acids of the full length cytokine. Particular embodiments disclosed herein provide the use of at least one cytokine, protein, or polypeptide, or at least one polynucleotide encoding a cytokine, protein, or polypeptide, that functions equivalently to any one of the cytokines selected from the group consisting of CCL5, TNFα, BMP2, Wnt, or a combination thereof.

In cases where a CCL5, TNFα, BMP2, or Wnt polynucleotide is utilized in methods of the disclosure, the polynucleotide may be at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:1, SEQ ID NO:2, SEQ ID NO:5, or SEQ ID NO:7, respectively. In embodiments where a CCL5, TNFα, BMP2, or Wnt polypeptide is utilized in methods of the disclosure, the polypeptide may be at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:3, SEQ ID NO:4, the polypeptide encoded by SEQ ID NO:6, or SEQ ID NO:8, respectively. In certain embodiments, the polypeptide may be a fragment of a polypeptide of SEQ ID NO:3, SEQ ID NO:4, the polypeptide encoded by SEQ ID NO:6, or SEQ ID NO:8, respectively; the fragment may be at least 25, 50, 75, 100, 125, 150, 200, 225, 250, 275, 300, 325, 350, 375, 400, 425, 450, 475, 500, and so forth.

In particular embodiments, the composition administered to the site in need is an agent that inhibits CCR5, such as maraviroc, aplaviroc, vicriviroc, or related agents. The composition may also include an agent that inhibits the function of CCL5, TNFα, BMP2, or Wnt. The agents may be antibodies, including neutralizing antibodies, humanized antibodies, scFvs, or a combination thereof. Examples of antibodies that target CCR5 include those described by Olson and Jacobson (Curr Opin HIV AIDS, 2010) which is incorporated by reference herein in its entirety. Examples of antibodies that target CCL5 include those described by Aldinucci et al (International Journal of Cancer, 2007) and Glass et al (The Journal of Immunology, 2004), which are incorporated by reference herein in their entirety. Examples of antibodies that target TNFα include those described by Chatterjea et al (F1000Res, 2013) and Williams et al (PNAS, 1992), which are incorporated herein in their entirety. Examples of antibodies that target BMP2 include those described by Su et al (JBC, 2007), which is incorporated herein in its entirety.

One skilled in the art can determine the therapeutically effective amount of composition to administer to a site in need in an individual. Determination of efficacy can be determined by monitoring the site that has been administered the composition, such as by X-ray imaging or a physical examination. In particular embodiments, the amount of composition used may be approximately at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 ng either in total or per kilogram of the individual to be administered. In specific embodiments, the amount of composition used may be approximately at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg either in total or per kilogram of the individual to be administered. In some embodiments, the amount of composition used may be approximately at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM of agent to be administered to the individual. In some embodiments, the administered concentration is or is about 10, 20, 25, 30, 40, 50, 60, 70, 75, 80, 90, 100, 110, 120, 125, 130, 140, or 150 ng/ml. Ranges may be or may be about 10-150, 10-140, 10-130, 10-125, 10-120, 10-110, 10-100, 10-90, 10-80, 10-75, 10-70, 10-60, 10-50, 10-40, 10-30, 10-20, 20-150, 20-140, 20-130, 20-130, 20-125, 20-120, 20-110, 20-100, 20-90, 20-80, 20-70, 20-60, 20-50, 20-40, 20-30, 30-150, 30-140, 30-130, 30-125, 30-120, 30-110, 30-100, 30-90, 30-80, 30-75, 30-70, 30-60, 30-50, 30-40, 40-150, 40-140-, 40-130, 40-125, 40-120, 40-110, 40-100, 40-90, 40-80, 40-70, 40-60, 40-50, 50-150, 50-140, 50-130, 50-125, 50-120, 50-110, 50-100, 50-90, 50-80, 50-70, 50-60, 60-150, 60-140, 60-130, 60-125, 60-120, 60-110, 60-100, 60-90, 60-80, 60-75, 60-70, 70-150, 70-140, 70-130, 70-125, 70-120, 70-110, 70-100, 70-90, 70-80, 70-75, 80-150, 80-140, 80-130, 80-120, 80-110, 80-100, 80-90, 90-150, 90-140, 90-130, 90-125, 90-120, 90-110, 90-100, 100-150, 100-140, 100-130, 100-125, 100-120, 100-110, 110-150, 110-140, 110-130, 110-125, 110-120, 120-150, 120-140, 120-130, 120-125, 130-150, 130-140, or 140-150 ng/ml.

The number of administrations may be determined by one skilled in the art. In some embodiments, a single administration may be used, while in further embodiments, multiple administrations are given to the individual.

### IV. Biocompatible Gel and/or Scaffold

In particular embodiments, the composition administered to the site in need may comprise a biocompatible gel and/or scaffold. Any biocompatible gel and/or scaffold may be used, wherein the gel and/or scaffold may enhance the efficacy of the cytokine or agent described above. The gel and/or scaffold may be comprised of matrigel, hydrogel, periosteum, hydroxyapatite, tricalcium phosphate, polystyrene, poly-1-lactic acid, polyglycolic acid, poly-dl-lactic-co-glycolic acid, collagen, proteoglycans, alginate-based substrates, chitosan, collagen-GAG, extracellular matrix, or a combination thereof. The gel and/or scaffold may be produced using any method known in the art. Gels and/or scaffolds that require being produced from an in vivo source, such as periosteum, may be from a source autologous, allogeneic, syngeneic, xenogeneic, or a combination thereof to the individual to be administered the composition at the site in need.

In particular embodiments, the cytokine(s) and/or agent(s) is/are combined with the biocompatible gel and/or scaffold in order to produce a new composition. The new composition may be used in the methods of the present disclosure. For example, CCL5 may be combined with matrigel to form a new composition that may be administered to a site in need, such as a bone break, in order to recruit P-SSCs to differentiate into osteoblasts and induce bone healing, while reducing the recruitment of OCPs, and thus reduce osteoclasts recruitment to the site in need.

### V. Pharmaceutical Preparations

Pharmaceutical compositions of the present disclosure comprise an effective amount of one or more cytokines, CCR5 inhibitors, TNFα inhibitors, stem cells or additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one cytokine, CCR5 inhibitor, TNFα inhibitor, or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The cytokine or CCR5 inhibitor or TNFα inhibitor may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The cytokine or CCR5 inhibitor or TNFα inhibitor may be formulated into a composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include at least one cytokine or CCR5 inhibitor or TNFα inhibitor, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, the cytokine or CCR5 inhibitor or TNFα inhibitor may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present disclosure, the cytokine or CCR5 inhibitor or TNFα inhibitor are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations hat are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the cytokine or CCR5 inhibitor or TNFα inhibitor may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound, comprising a cytokine or CCR5 inhibitor or TNFα inhibitor may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VI. Kits

Any of the compositions described herein may be comprised in a kit. In a non-limiting example, a cytokine or CCR5 inhibitor or TNFα inhibitor, lipid, and/or additional agent, may be comprised in a kit. The kits will thus comprise, in suitable container means, a cytokine or CCR5 inhibitor or TNFα inhibitor and a lipid, and/or an additional agent of the present disclosure.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the cytokine or CCR5 inhibitor or TNFα inhibitor, lipid, additional agent, and any other reagent containers in close confinement for commercial sale. Such containers may include injection or blow-molded plastic containers into which the desired vials are retained.

Therapeutic kits of the present disclosure are kits comprising a cytokine protein, polypeptide, peptide, inhibitor, gene, vector and/or other CCR5 effector. Such kits will generally contain, in suitable container means, a pharmaceutically acceptable formulation a cytokine protein, polypeptide, peptide, domain, inhibitor, and/or a gene and/or vector expressing any of the foregoing in a pharmaceutically acceptable formulation. The kit may have a single container means, and/or it may have distinct container means for each compound.

When the components of the kit are provided in one and/or more liquid solutions, the liquid solution is an aqueous solution, with a sterile aqueous solution being particularly preferred. The cytokine or CCR5 inhibitor or TNFα inhibitor compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the cytokine protein, gene and/or inhibitory or CCR5 inhibitor formulation are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate cytokine protein and/or gene composition or CCR5 inhibitor within the body of an animal. Such an instrument may be a syringe, pipette, forceps, and/or any such medically approved delivery vehicle.

### VII. Examples

The following examples are included to demonstrate particular embodiments of the disclosure. It should be appreciated by those of skill in the art that the techniques disclosed in the examples that follow represent techniques discovered by the inventor to function well in the practice of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### The Mx1 and ASMA Combination Selectively Labels Endogenous P-SSCs

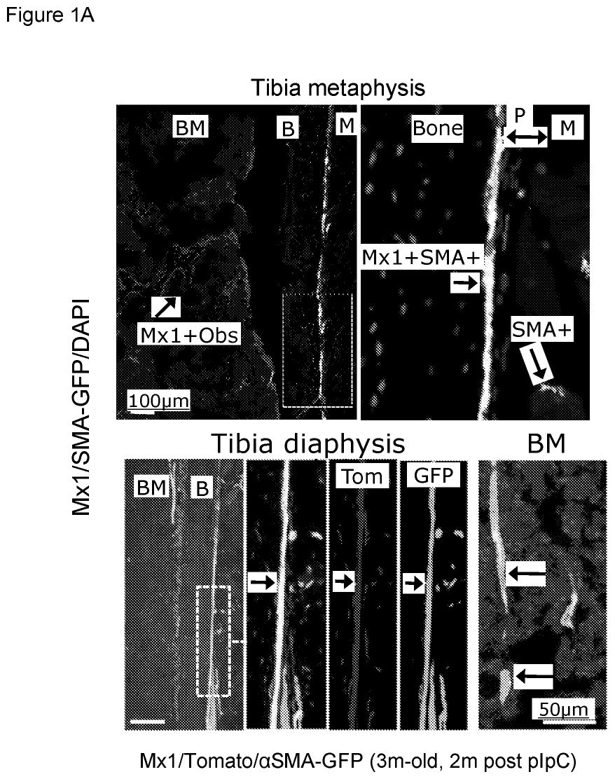

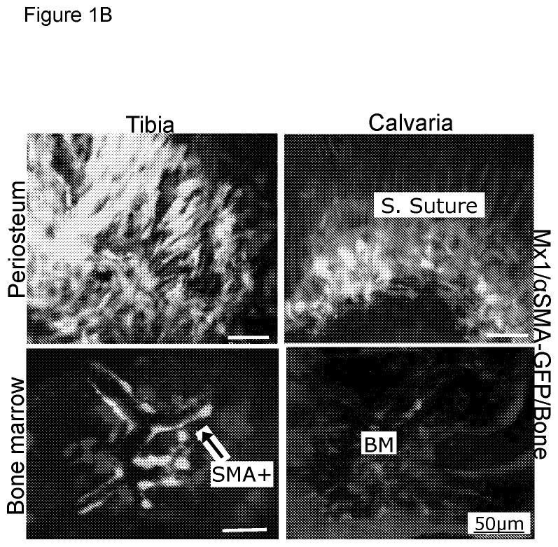

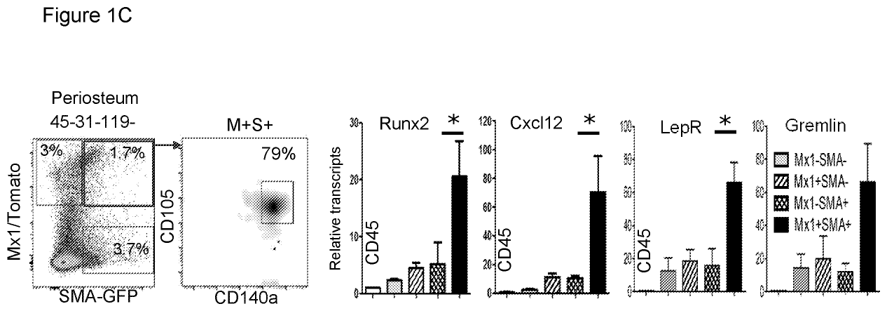

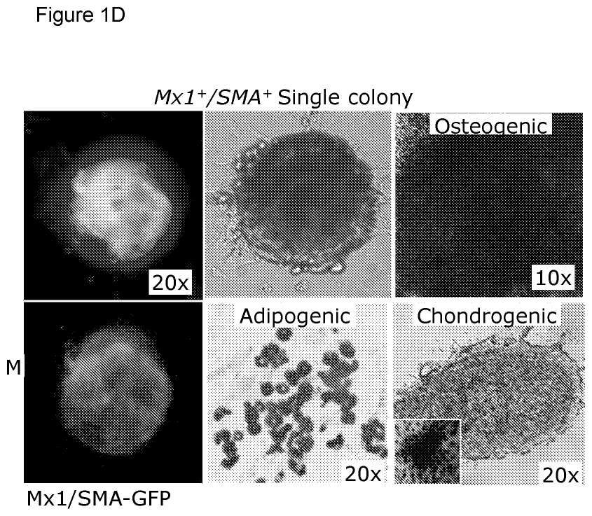

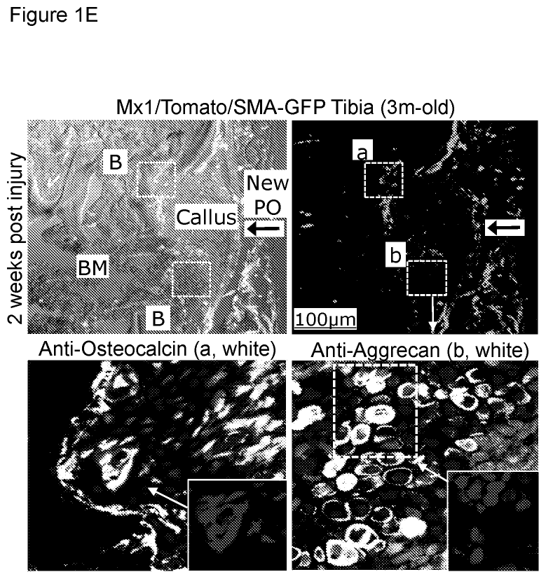

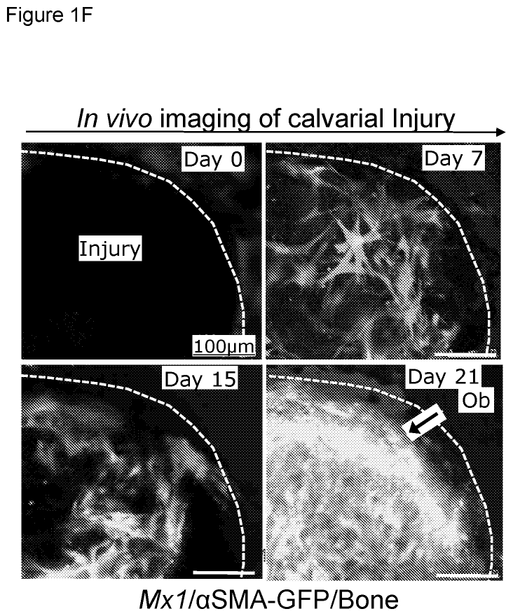

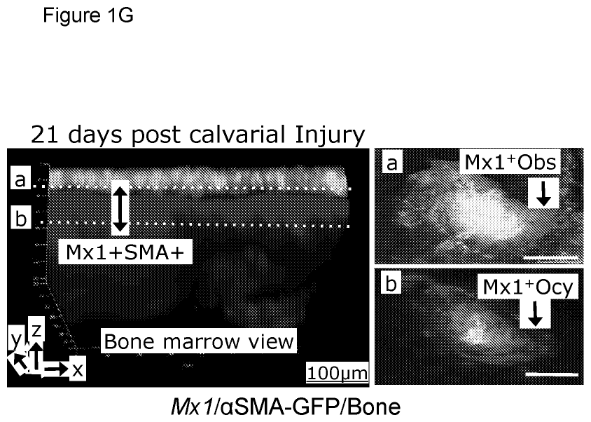

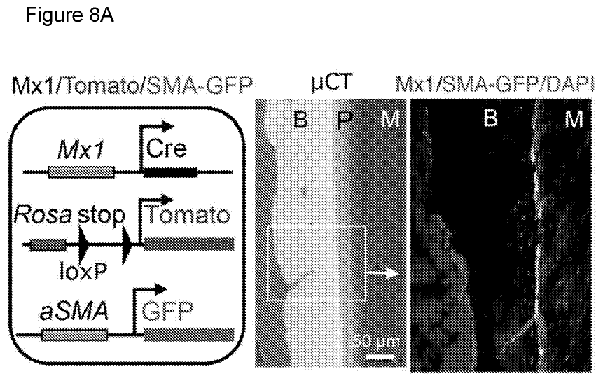

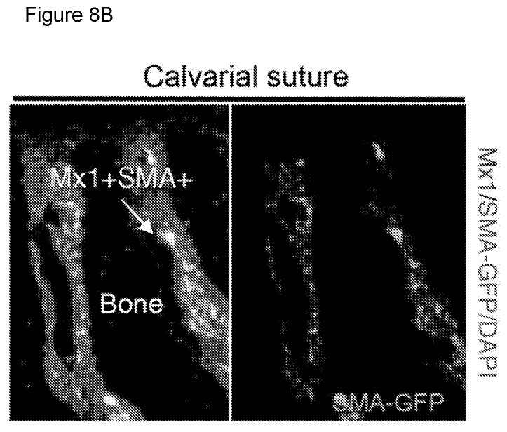

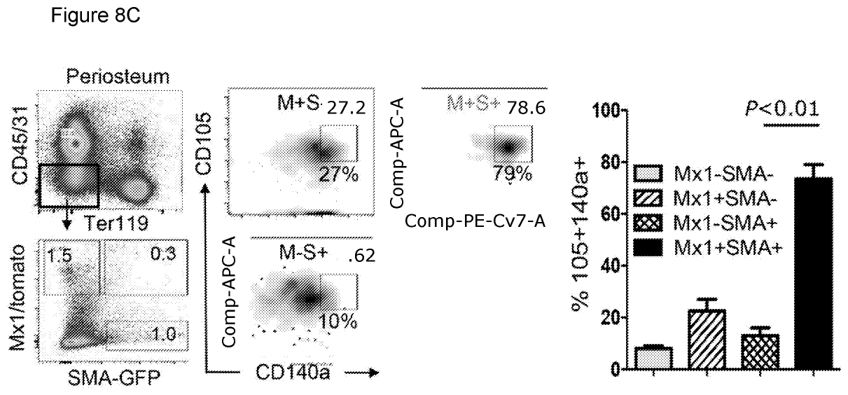

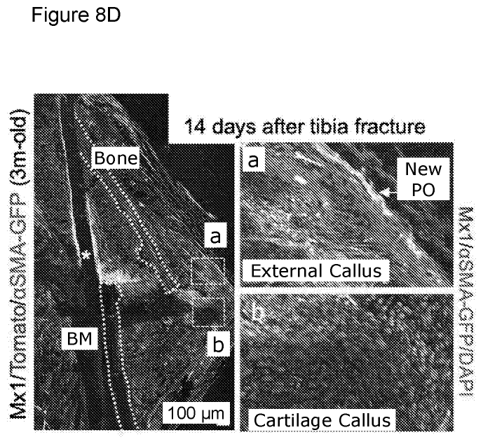

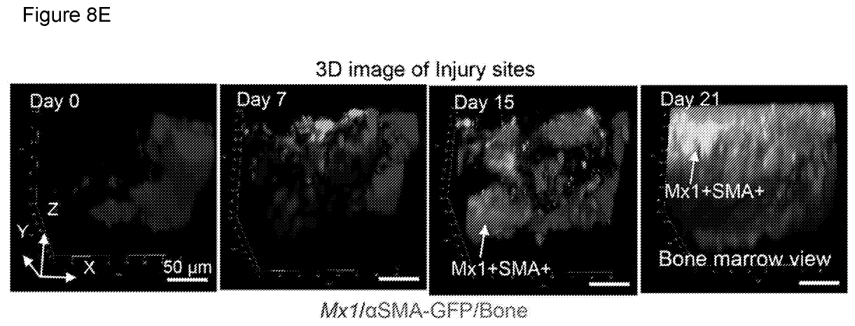

The origin of new osteoblasts, particularly at fracture sites, has long been an important question for biologists (Schindeler et al., 2008). Recent studies revealed that a subset of periosteal cells has skeletal progenitor function and plays an important role in bone repair (van Gastel et al., 2012). However, there is no known P-SSC specific marker, making the in vivo origin and regulatory mechanism of these cells elusive. Studies have reported that Mx1 labels endogenous osteogenic stem/progenitor cells that can maintain the majority (>80%) of the mature osteoblast population over time in the adult mouse (Park et al., 2012a). It was also found that these cells are present in the periosteum and supply new osteoblasts for bone healing. Recently, several models have been developed to label P-SSCs; however, one limitation of these models, like the Mx1 model, is that both P-SSCs and multiple cell types are labeled. Therefore, to further define a model enabling selective labeling of P-SSCs, Mx1-Cre⁺Rosa26-Tomato⁺ mice were crossed with other mesenchymal stem cell (MSC) reporter mouse lines, including αSMA-GFP (Grcevic et al., 2012), Nestin-GFP (Mendez-Ferrer et al., 2010), and CXCL12-GFP (Omatsu et al., 2010). Among the tested combinations, immunohistochemistry of trigenic Mx1-Cre⁺Rosa26-Tomato^{±}αSMA-GFP⁺ (Mx1/Tomato/αSMA-GFP) mice after a wild-type BM transplant showed that the Mx1 and αSMA-GFP combination selectively labels a subset of periosteal cells (FIG. 1A). To test whether Mx1/αSMA-GFP double labeling (Mx1⁺αSMA⁺) can specify P-SSCs, and to define their in vivo location in the periosteum and in the BM, polyinosinic:polycytidylic acid (pIpC) was administered to trigenic Mx1/Tomato/αSMA-GFP mice (25 mg/kg every other day for 10 days) and transplanted wild-type BM into irradiated Mx1/Tomato/SMA-GFP mice to eliminate Mx1⁺ hematopoietic cells. Four to six weeks later, histological and in vivo image analysis of Mx1/Tomato/αSMA-GFP mouse calvaria and tibia revealed that most Mx1⁺αSMA⁺ (Tomato⁺GFP⁺) cells reside in the periosteum and calvarial sutures (a known niche for craniofacial SSCs (FIG. 8B)) (Zhao et al., 2015), but are nearly undetectable in the BM (FIG. 1A, FIG. 1B, and FIG. 8A). Most BM-SSCs, and their colony-forming units-fibroblast (CFU-F) activity, are enriched in the non-hematopoietic (CD45⁻Ter119⁻CD31⁻) Sca1⁺CD140a⁺ (Chan et al., 2009; Morikawa et al., 2009), CD140a⁺CD51⁺ (Pinho et al., 2013) or CD105⁺CD140a⁺ fractions (Park et al., 2012a). Interestingly, fluorescence-activated cell sorting (FACS) analysis of the periosteum of Mx1/Tomato/αSMA-GFP mice showed that periosteal cells form a heterogeneous population and that ˜30% of Mx1⁺ periosteal cells are αSMA-GFP positive, and approximately 80% of Mx1⁺αSMA⁺ periosteal cells express SSC immunophenotypic markers (CD105⁺CD140a⁺) with higher expression of SSC transcripts, including Grem1, LepR, and Cxcl12 (FIG. 1C). By contrast, only ˜10% of αSMA⁺ cells and ˜27% of Mx1⁺ cells from the CD45⁻CD31⁻Ter119⁻ fraction are CD105⁺CD140a⁺, with a ˜5-fold lower expression of SSC transcripts (FIG. 1C and FIG. 8C). When cultured at the single-cell level, Mx1⁺αSMA⁺ periosteal cells can form hemisphere colonies with osteogenic, chondrogenic, and adipogenic differentiation potential in vitro, demonstrating that Mx1⁺αSMA⁺ periosteal cells exhibit ex vivo SSC characteristics and that the Mx1 and αSMA-GFP combination, but not αSMA-GFP alone, further specifies endogenous P-SSCs in vivo (FIG. 1D).

Given that periosteal progenitor cells are reported to give rise to bone and cartilage in response to injury (Colnot, 2009), It was examined whether Mx1⁺αSMA⁺ periosteal cells contribute to the formation of osteoblasts during injury repair in vivo. To define the origin and dynamics of the osteoblasts located at injury sites, drill-hole defects (˜1 mm diameter) were generated in the tibia, using a needle (20 G), in Mx1/Tomato/αSMA-GFP dual reporter mice. Histology of tibia injury at day 14 revealed that Mx1⁺αSMA⁺ periosteal cells repopulate the outer layer of the callus (FIG. 1E, New PO) and supply the majority of callus-forming cells (FIG. 1E, Callus). It was previously reported that Mx1⁺ stem/progenitors in the BM exhibit mainly osteogenic potential, with little adipogenic and no chondrogenic potential in postnatal bone maintenance (Park et al., 2012a). Interestingly however, immunofluorescence staining of chondrocytes (anti-aggrecan) and osteoblasts (anti-osteocalcin) in the injury revealed that Mx1⁺αSMA⁺ periosteal cells contribute to 21±3% of chondrocytes in the cartilage intermediate, and over 80% of new osteoblasts within the callus, implicating that they differ from Mx1⁺ BM-SSCs (FIG. 1E). It was consistently found that Mx1⁺αSMA⁺ cells mainly contributed to callus formation at 14 days after a transverse tibial fracture (FIG. 8D). When Mx1⁺αSMA⁺ periosteal cells in this model were tracked during bone repair of injured calvaria in vivo, these cells newly appeared at the injury site and repopulated on days 7 and 15 (FIG. 1F, FIG. 8E). In addition, 21 days after injury, in vivo imaging with 3D reconstitution of Z-stack images showed that the endosteal space of the injury region was filled with differentiated Mx1⁺αSMA⁺ cells, while Mx1⁺αSMA⁺ cells exclusively resided in the outer surface and formed new periosteum at the injury site (FIG. 1F & FIG. 8D). Additionally, 21 days post-injury, Mx1⁺αSMA⁺ cells near the injured bone surface became Mx1⁺αSMA⁺ osteoblasts and osteocytes in newly formed bones (FIG. 1G). Of note, Mx1⁺ BM cells near the injury site showed no observable activation. These data support the idea that Mx1⁺αSMA⁺ periosteal cells include endogenous P-SSCs with a multi-lineage differentiation potential, and that the migration and proliferation of Mx1⁺αSMA⁺ P-SSCs supply the osteoblasts participating in bone healing.

### Example 2

### Mx1+P-SSCs are the Major Source of New Osteoblasts in Bone Healing In Vivo

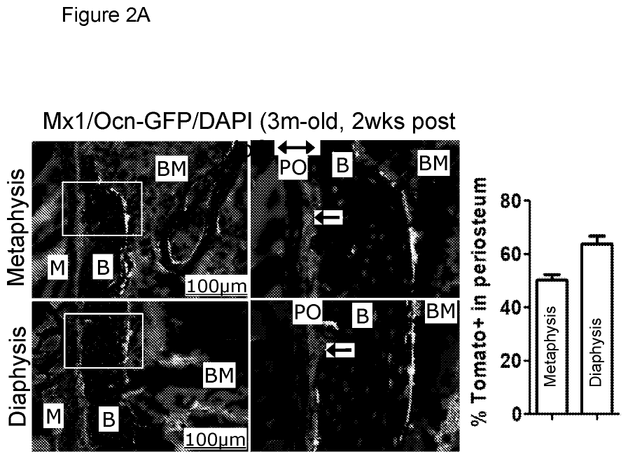

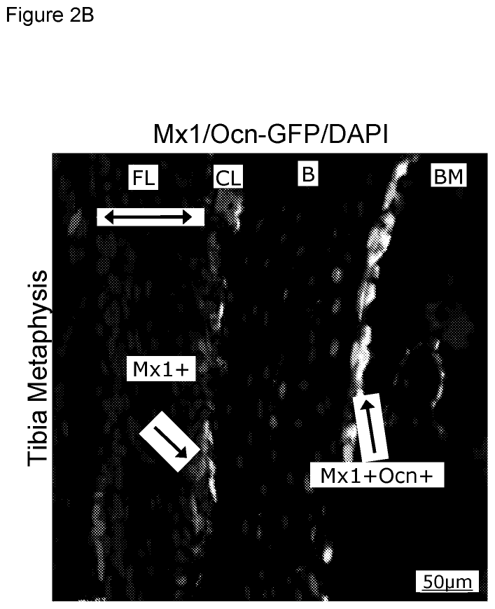

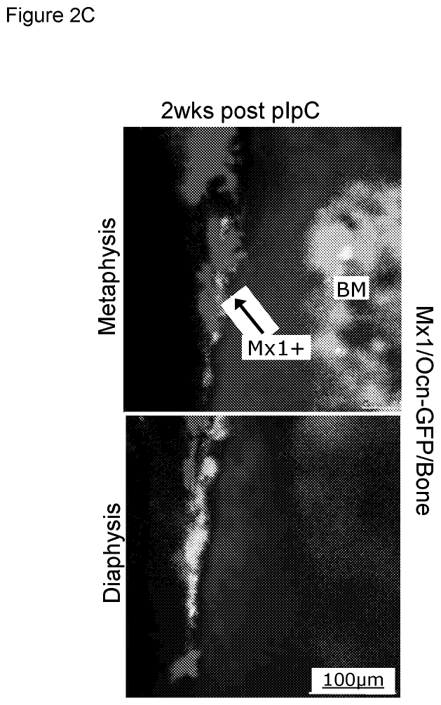

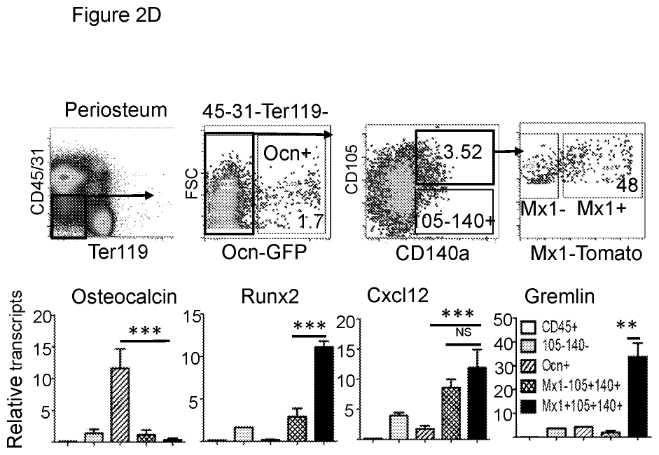

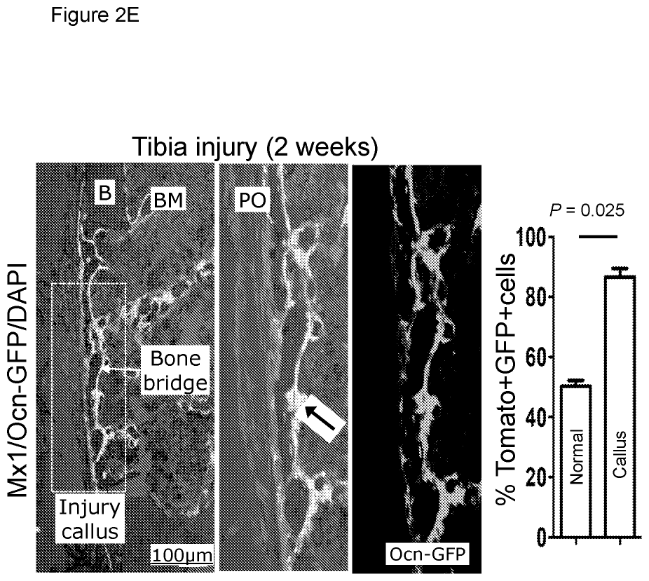

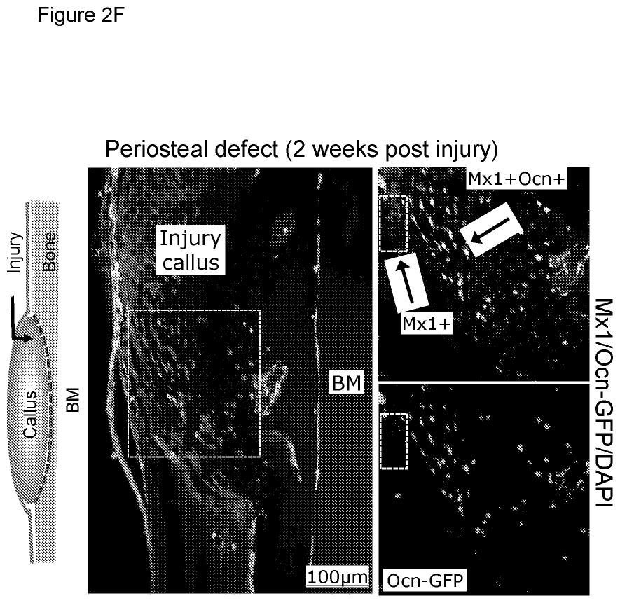

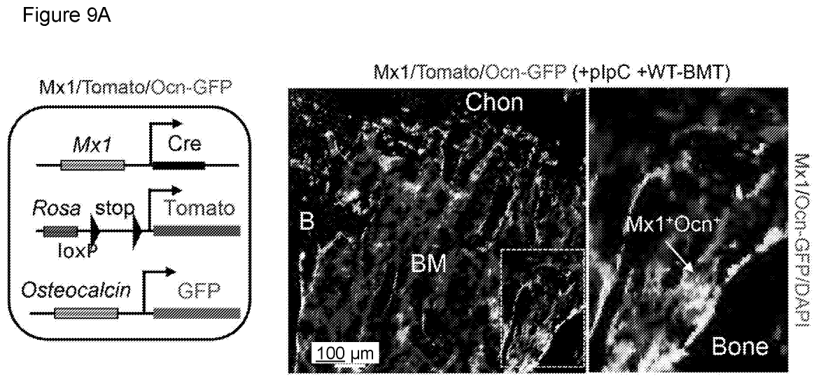

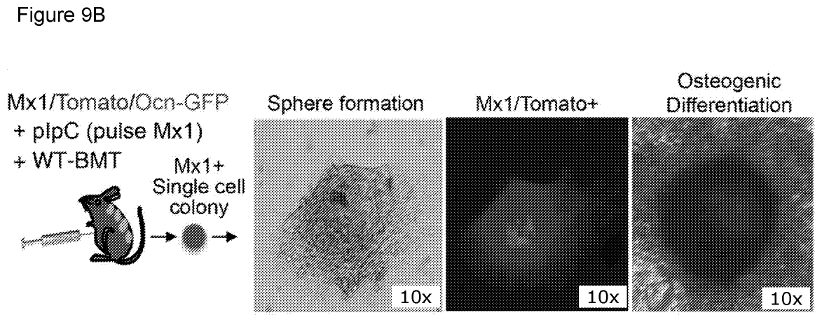

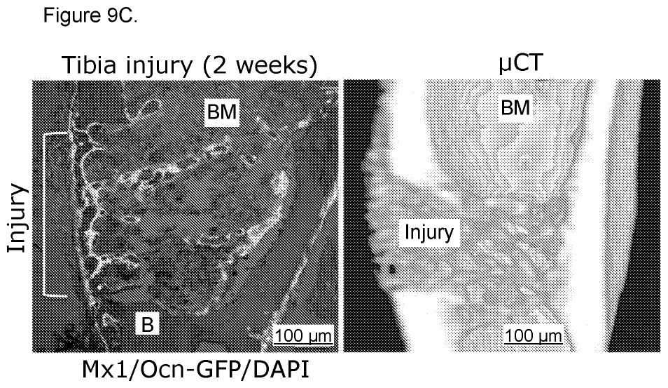

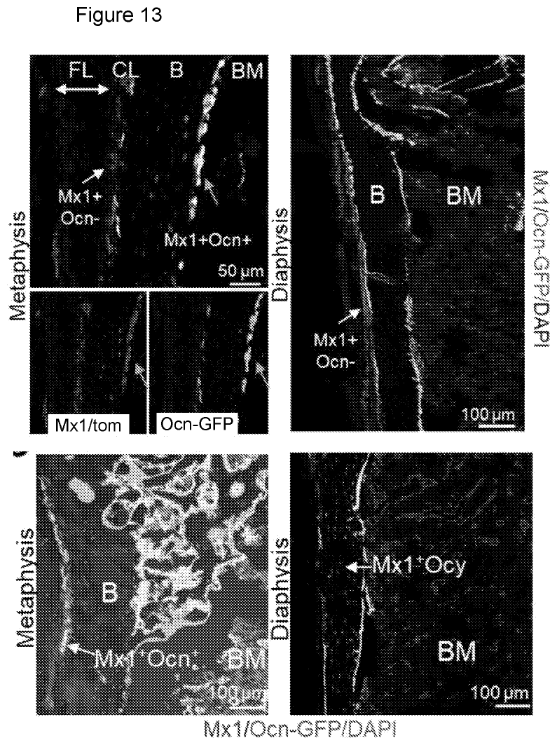

It is possible that the periosteum contains Mx1⁻ P-SSCs that can contribute to new osteoblasts at injury sites. Therefore, to distinguish between P-SSCs and mature osteoblasts in the periosteum and to improve specific cell quantitation during bone healing, trigenic Mx1-Cre⁺Rosa26-Tomato⁺ osteocalcin-GFP⁺ (Mx1/Tomato/Ocn-GFP) reporter mice were generated. In this model, when Mx1+P-SSCs (Tomato+) differentiate into mature Ocn-GFP⁺ osteoblasts they express Tomato and GFP, while mature osteoblasts from Mx1⁻ P-SSCs express GFP alone (Park et al., 2014). In vivo imaging and immunohistochemistry analysis of Mx1/Tomato/Ocn-GFP femurs and tibias revealed that the induction of Mx1-Cre activity can label distinct periosteal stem/progenitor cells without detectable Ocn-GFP expression in the metaphysis (50±4%) and diaphysis (64±6%) (FIG. 2A). In contrast to a widely mixed distribution of Mx1⁺ progenitors (Tomato⁺) and osteoblasts (Tomato⁺GFP⁺; ˜50-60% of endosteal osteoblasts) in trabecular bones (FIG. 2B, FIG. 13), these Mx1⁺ P-SSCs exclusively exist in the cambial layer (a site reported by others to be the location of skeletal progenitors) of the calvaria and femur (FIG. 2B & FIG. 2C). To determine that Mx1±Ocn⁻ periosteal cells contribute to normal periosteal bone growth, the percentage of Mx1⁺Ocn⁻ periosteal osteoblasts and osteocytes in the tibia of Mx1/Tomato/Ocn-GFP mice at 2, 8, and 16 weeks after pIpC treatment. The number of Mx1⁺Ocn⁻ periosteal osteoblasts and osteocytes in cortical bone was markedly increased in 7-month-old mice (from −18% at 2 weeks to ˜80% at 16 weeks post pIpC) supporting that Mx1⁺Ocn⁻ periosteal cells are the major source of newly generated periosteal osteoblasts and osteocytes in postnatal bones. Immunophenotypic analysis of periosteal cells from collagenase-digested tibia collected from Mx1/Tomato/Ocn-GFP mice revealed that Mx1⁺ P-SSCs accounted for 48% of the non-hematopoietic CD105⁺CD140a⁺ fraction of periosteal cells and formed clonogenic mesenspheres with in vitro osteogenic differentiation potential (FIG. 9B). Next, to test whether P-SSCs are enriched in Mx1⁺ periosteal cells, Mx1⁻ and Mx1⁺ cells were sorted within the SSPC (CD45⁻CD31⁻TER119⁻CD105⁺CD140a⁺) population from the periosteum. Gene expression analysis of these fractions and the control fractions revealed that the non-hematopoietic Mx1⁺CD105⁺CD140a⁺ population expressed significantly higher levels of SSC markers, including Grem1, LepR, Cxcl12, and Runx2, but no detectable levels of Ocn (a mature osteoblastic marker) compared with Mx1⁻ progenitors and other populations (FIG. 2D), indicating that P-SSC properties are exclusively enriched in Mx1⁺ periosteal cells.

To test whether Mx1⁺ P-SSCs are the main source of new osteoblasts during bone repair in vivo, drill-hole defects (˜1 mm diameter removal of bone and periosteum) were generated in the tibial diaphysis of Mx1/Tomato/Ocn-GFP mice. A drill-hole defect can potentially be a model of bone healing without compromising tissue architecture (endosteum vs periosteum) and achieve better quantification of stem cell responses (He et al., 2011). Immunohistochemistry analysis of the bone defect revealed that Mx1⁺ P-SSCs reconstitute the majority (˜80%) of outer callus-forming cells with new periosteum (Tomato+), and >70% of the bone-forming osteoblasts (Tomato⁺GFP⁺) surrounding new bone bridges (FIG. 2E & 9C). Next, to investigate the specific injury response of periosteal cells, a bone defect in the periosteum was generated and the outer surface of cortical bone, without BM damage, in the tibia of Mx1/Tomato/Ocn-GFP mice. Two weeks post-injury, there was consistently observed robust callus formation with over 90% contribution of Mx1⁺ P-SSCs to new osteoblasts (GFP⁺) in the injury callus (FIG. 2F). Taken together, these data show that Mx1 can label stem/progenitor cells in the periosteum and that the activation of Mx1⁺ P-SSCs upon cortical bone injury is sufficient to induce the injury response and osteogenesis.

### Example 3

### Mx1⁺αSMA⁺ P-SSCs are Long-Term Repopulating Progenitors Required for Bone Healing

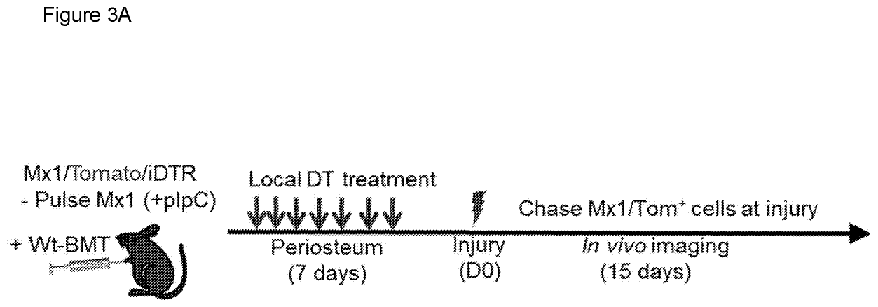

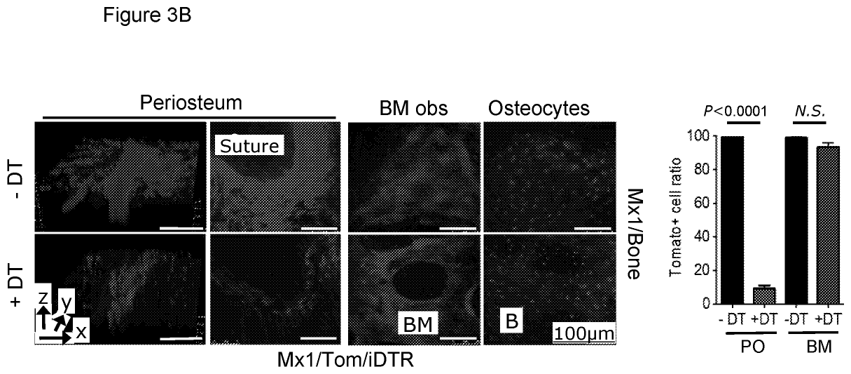

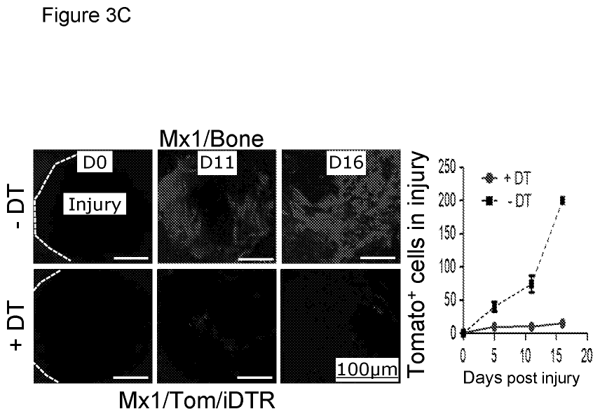

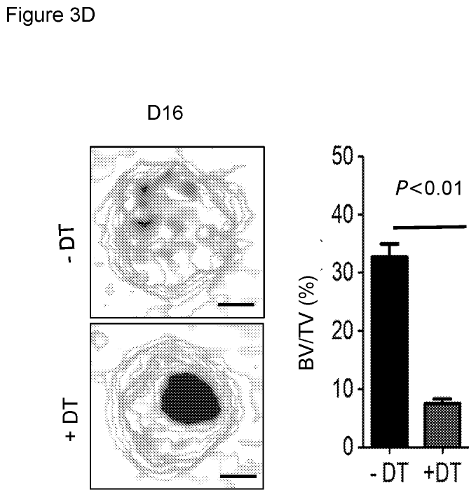

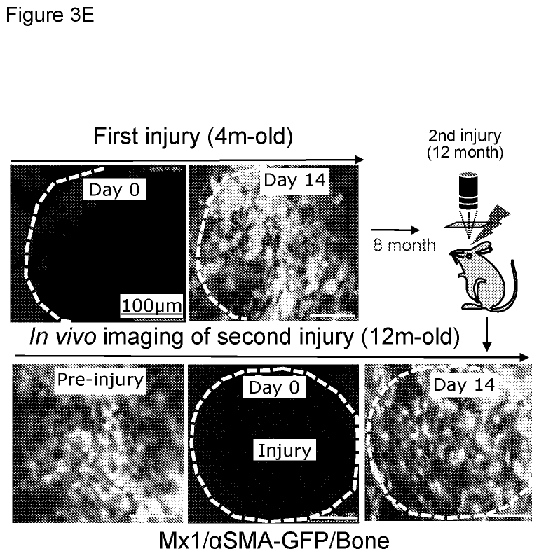

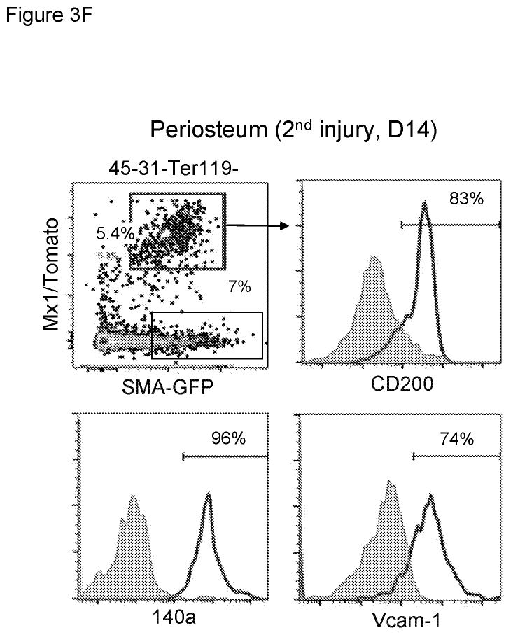

SSCs are known to be present in the BM (Worthley et al., 2015) and may contribute to bone healing if the periosteum is defective or removed. To determine if Mx1⁺ periosteal cells are essential for bone healing, an inducible deletion model was developed by intercrossing Mx1-Cre/Rosa26-Tomato mice with Rosa26-DTR mice (Park et al., 2012a) and the requirement for Mx1⁺ periosteal cells in bone healing was tested. To restrict the conditional deletion of Mx1⁺ cells to the periosteum, local surface administration of diphtheria toxin (DT) (20 μL, 1 μg/mL) was used to cover an area with ˜1 cm of diameter at the junction of the sagittal and coronal sutures (FIG. 3A). Treatment of the periosteum with DT for seven days prior to injury reduced the number of Mx1⁺ periosteal cells in more than 90%, while Mx1⁺ cells in the BM and in the distal periosteum remained unchanged (FIG. 3B). Under these conditions, sequential imaging of injury repair for 16 days revealed that the DT-mediated local deletion of Mx1⁺ periosteal cells completely inhibits the recruitment and proliferation of Mx1⁺ osteogenic cells at the injury site (FIG. 3C). It was also confirmed that local DT treatment significantly delays both calvaria and tibia bone healing by micro-computerized tomography (μCT) analysis (FIG. 3D). Since Mx1-labeled osteoblasts and progenitors in the BM near the injury site remained intact, and no detectable changes in their population or position occurred during injury healing, the new Mx1+ cells within the bone injury were highly likely to be derived from the periosteum. These data are consistent with Mx1⁺ periosteal cells being necessary for osteogenesis and bone healing.

An important characteristic of stem cells is their long-term repopulation capability after multiple rounds of injuries (Sacchetti et al., 2007). Given that a subset of Mx1⁺αSMA⁺ periosteal cells remains on the outer layer of the callus even after bone healing, the stem cell function of these cells was tested by examining the long-term repopulation ability of Mx1⁺αSMA⁺ periosteal cells and their contribution to bone repair after several rounds of injuries. Consecutive intravital imaging of the primary injury sites of Mx1/Tomato/αSMA-GFP mice at ˜4 months of age (FIG. 1D), and again at ˜10 months of age, revealed complete bone recovery and that Mx1⁺αSMA⁺ periosteal cells returned to pre-injury homeostatic levels (FIG. 3E, Pre-injury). When the secondary injuries were generated near the primary injury sites and Mx1⁺αSMA⁺ periosteal cells were further traced at the secondary injury sites, they rapidly repopulated and contributed to over 80% of osteoblasts, similar to their response to the primary injury (FIG. 3E, D14). These data indicate that the Mx1 and αSMA-GFP combination labels a long-term repopulating P-SSC subset that sustains itself while durably supplying the majority of new osteoblasts in bone injury over time in the adult mouse.

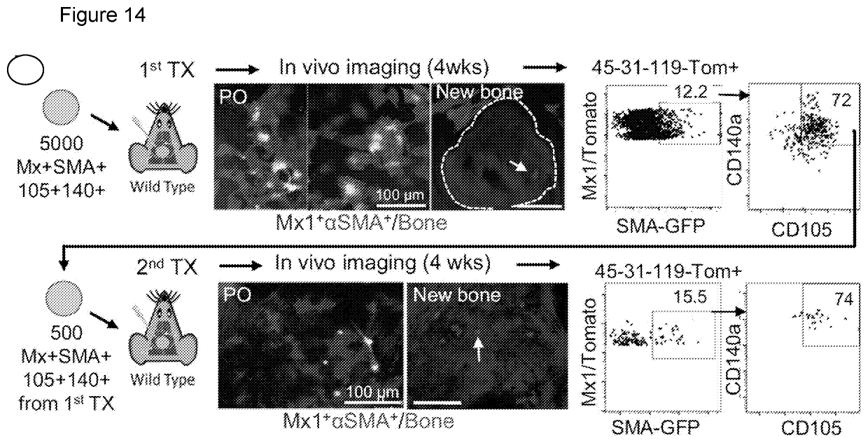

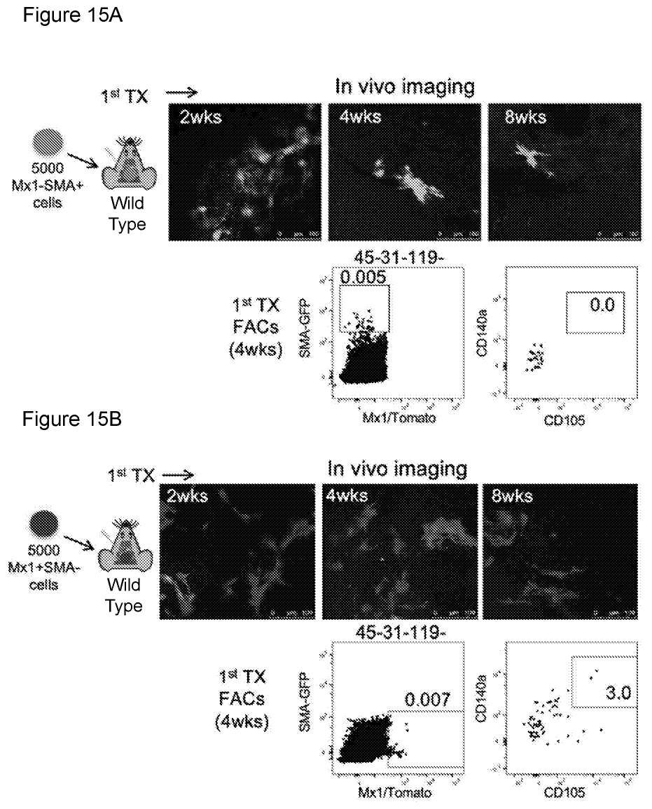

To better define the long-term repopulation characteristics and to exclude the possibility that Mx1⁺αSMA⁻ periosteal cells include P-SSCs and become Mx1⁺αSMA⁺ cells in bone injury, we performed serial transplantations of these cells (FIG. 14). A total of 5,000 Mx1⁺αSMA⁺CD140a⁺CD105⁺, Mx1⁺αSMA⁻, and Mx1⁺αSMA⁺ cells from the periosteal CD45⁻CD31⁻Ter119⁻ fraction were sorted and transplanted with Matrigel onto the calvaria injury site of a WT mouse. We reasoned that this P-SSC transplantation onto a periosteal injury site is more physiologically relevant compared to the previously described SSC transplantation into kidney capsules (Chan et al., 2015) or mammary fat pads (Debnath et al., 2018). In vivo imaging and FACS analysis at 4 weeks after the first round of transplantation showed that the transplanted Mx1⁺αSMA⁺ cells proliferated and differentiated into Mx1⁺ osteoblasts and osteocytes that were imbedded within the newly formed bone tissue (FIG. 14, top arrow, New bone). A subset of the engrafted cells repopulated and maintained the Mx1⁺αSMA⁺ population in the periosteum (12%-15% of all transplanted Mx1⁺ cells). The majority of the Mx1⁺αSMA⁺ cells (˜72%) preserved the expression of SSPC surface markers (CD140a⁺CD105⁺) (FIG. 3C, top). When these Mx1⁺αSMA⁺ CD140a⁺CD105⁺ cells (˜500 cells/mouse) were re-transplanted and analyzed at 4 weeks after the second transplantation, they displayed both intact repopulation of double positive cells (FIG. 14, bottom PO, ˜15% of transplanted cells) with high CD140a⁺CD105⁺ expression (˜74%) and their osteoblast and osteocyte differentiation (FIG. 14, bottom arrow, New bone). However, neither transplanted Mx1⁺αSMA⁺ nor Mx1⁻αSMA⁺ cells did not show such repopulation, and there was no detectable stem cell marker expression (FIG. 15), demonstrating that the key characteristics of SSCs—in vivo long-term repopulation and differentiation—are highly enriched in the Mx1⁺αSMA⁺ periosteal cells.

### Example 4

### Mx1⁺ Periosteal Cells, Distinct from Nestin-GFP+BM-SSCs, Supply New Osteoblasts in Fracture Healing

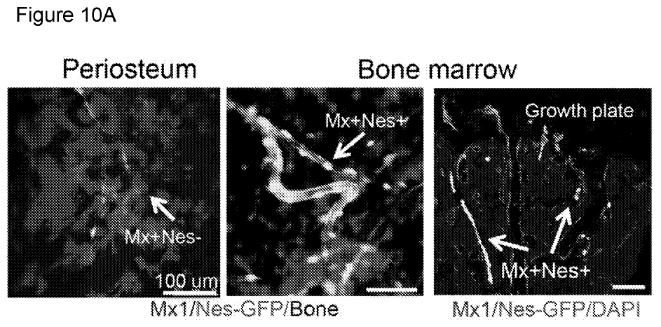

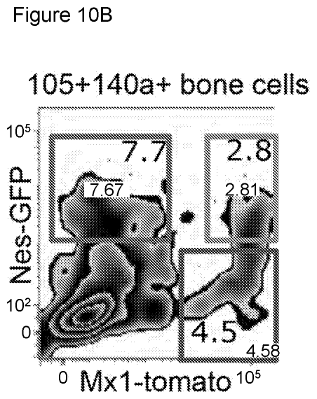

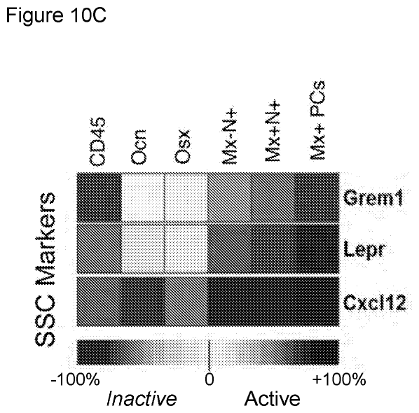

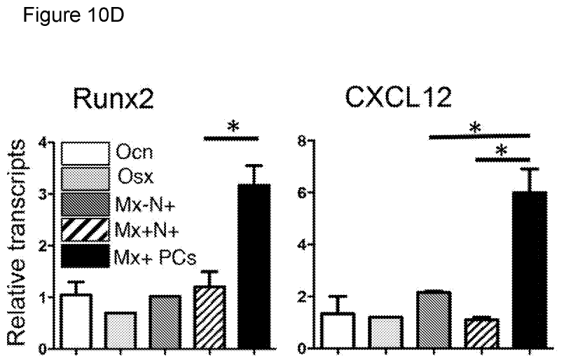

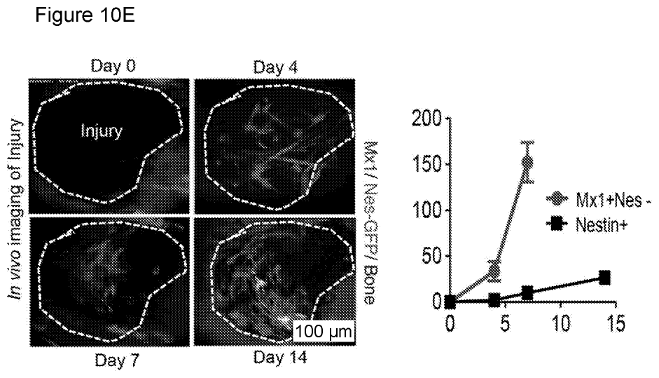

BM-SSCs were reported to localize in perivascular regions with Nestin-GFP expression (Mendez-Ferrer et al.). In addition, the CD31⁻Nestin-GFP⁺ perivascular reticular cells are LepR-Cre descendants and contribute to postnatal bone regeneration (Kunisaki et al., 2013). Mx1⁺ cells have been shown to overlap with a subset of Nestin⁺ populations in the BM (Park et al., 2012a), and therefore researchers developed the trigenic Mx1-Cre⁺Rosa26-Tomato⁺Nestin-GFP+(Mx1/Tomato/Nestin-GFP) dual reporter mouse and tested if Mx1 and Nestin double labeling can distinguish BM-SSCs from P-SSCs and their contributions to osteolineage cells in bone injury. In vivo imaging and FACS analysis of these mice revealed that there are two distinct cell populations (Mx1⁺Nestin⁺; ˜27% and Mx1⁻Nestin⁺; ˜73%) within Nestin⁺ perivascular cells, whereas Mx1⁺ periosteal cells are exclusively Nestin negative (FIG. 10A & FIG. 10B). To determine if marker expression was different between P-SSC and BM-SSC populations, Affymetrix-based global gene expression analysis with FACS-sorted cells were performed: (1) CD105⁺CD140a⁺Mx1⁺Ocn⁻ periosteal cells from Mx1/Tomato/Ocn-GFP mouse periosteum (FIG. 2D, Mx1+), (2) Mx1⁺Nestin⁺ and (3) Mx1⁻Nestin⁺ cells within the non-hematopoietic CD105⁺CD140a⁺ population from Mx1/Tomato/Nestin-GFP mouse bones and control cells, including (4) CD45⁺ hematopoietic cells, (5) Osteocalcin⁺ (Ocn⁺), and (6) Osterix⁺ (Osx⁺) cells from Osteocalcin-GFP and Osterix-GFP mice, respectively. Interestingly, the expression levels of SSC markers, including Grem1, LepR, and CXCL12, were much higher in Mx1⁺Ocn⁻ periosteal cells compared to Nestin⁺ BM perivascular cells and control cells. The highest expression level of Runx2 and CXCL12 was confirmed in Mx1⁺ periosteal cells by reverse transcription polymerase chain reaction RT-PCR (FIG. 10C & FIG. 10D). Mx1⁺Nestin⁺ (Tomato⁺GFP⁺), Mx1⁺Nestin⁻ (Tomato⁺), and Nestin⁺ (GFP⁺) cells were tracked at the bone injury site using intravital imaging and it was found that Mx1⁺Nestin⁻ periosteal cells predominantly appeared at the injury site at 2-5 days (>95%) and subsequently increased in number. In marked contrast, few Mx1⁺Nestin⁺ (2-3%) or Mx1⁻Nestin⁺ (˜1%) cells were observed at the injury site throughout the repair process (FIG. 10E). Taken together, these results demonstrate that Mx1⁺ subsets in the periosteum have SSC properties, respond to injury, and supply new osteoblasts.

### Example 5

### Mx1⁺aSMA⁺ P-SSCs have a Unique Migratory Mechanism Regulated by Ccr5 In Vivo

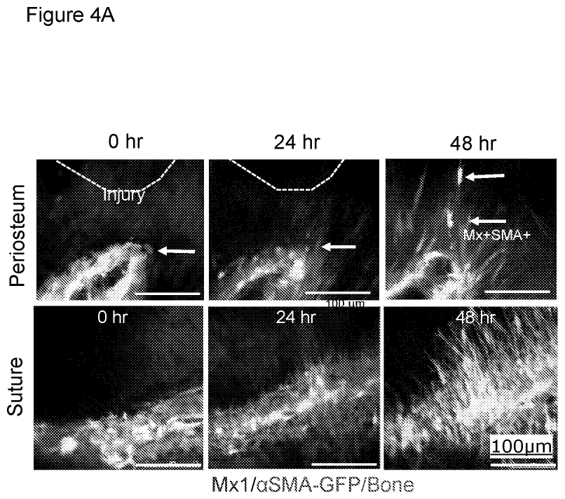

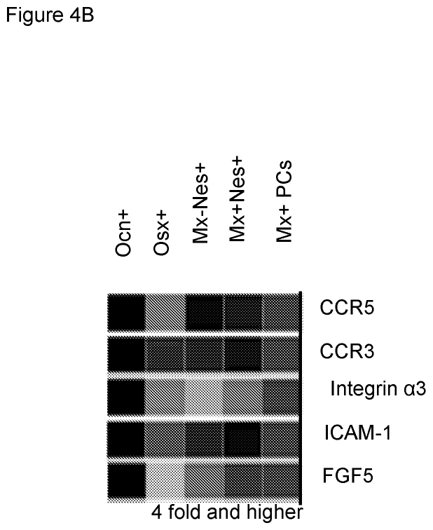

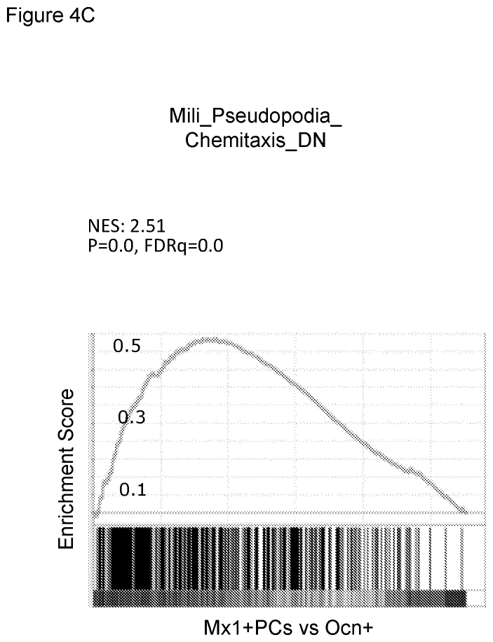

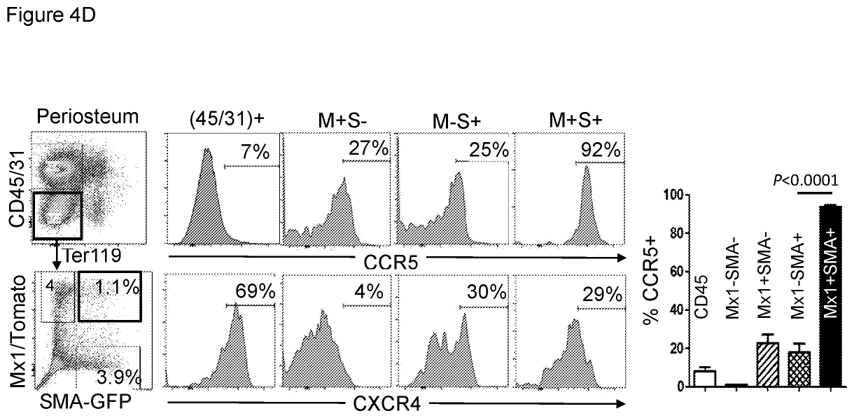

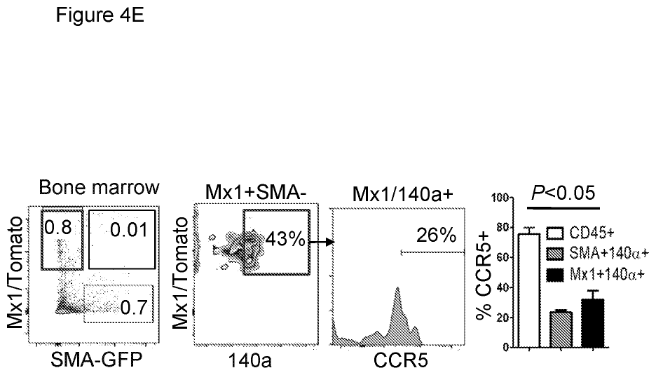

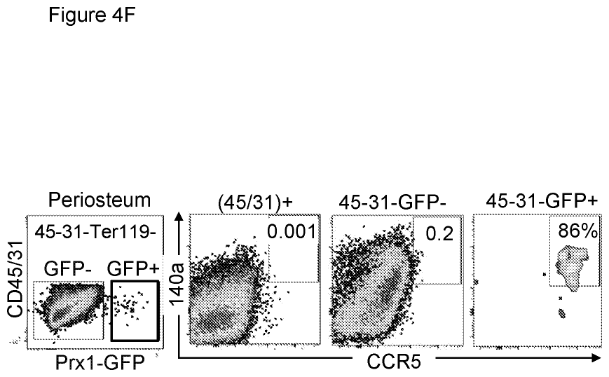

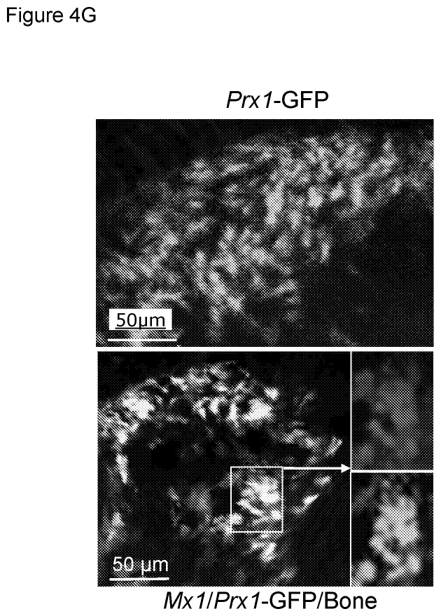

Skeletal progenitors have been proposed to have migratory or circulatory potential. However, there is no in vivo evidence for the endogenous migratory mechanism of P-SSCs. In particular, how P-SSCs are signaled to respond to injury and how they migrate and form fracture-repairing osteoblasts is essentially unknown. To test whether P-SSCs migrate toward the injury site in vivo, continuous Z-stack imaging of individual Mx1⁺αSMA⁺ P-SSCs was performed and sequentially scanning was performed on the detailed structures of the periosteum, bone matrix, and BM near the injury sites immediately (0), 24, and 48 hours after injury (FIG. 4A). Multiple Mx1⁺αSMA⁺ P-SSCs appeared to move toward the injury site (FIG. 4A, arrows), and, which was confirmed by Z-stack imaging. However, in agreement with FIG. 3D, the endosteal and vessel-associated Mx1⁺ cells were not observed to shift their position, indicating the presence of a unique migratory mechanism of Mx1⁺αSMA⁺ P-SSCs and suture cells.

Many growth factors and cytokines are known to enhance the migration and proliferation of in vitro-expanded MSCs (Einhorn and Gerstenfeld, 2015; Schindeler et al., 2008). However, there is no direct evidence of the effect of these molecules on the in vivo migration potential of endogenous SSCs. molecules involved in P-SSC migration were examined by Affymetrix-based global gene expression analysis of Mx1⁺Ocn⁻ periosteal cells compared to more differentiated cells and Nestin⁺ BM cells (FIG. 10C). Interestingly, It was found that two specific CC chemokine receptors, CCR3 and CCR5, are highly expressed in Mx1⁺ periosteal cells (FIG. 4B). It was also found that there was increased expression of genes involved in MSC migration and proliferation, including Integrin α3, Icam1, and FGF5. Consistently, Gene Set Enrichment Analysis (GSEA) showed a significant upregulation of the filopodia-associated migratory gene set (Gene Set: MILI_PSEUDOPODIA_CHEMOTAXIS_DN) in Mx1⁺ periosteal cells compared to more mature osteoblasts (FIG. 4C). Subsequent FACS and real-time PCR analysis determined that almost all Mx1⁺αSMA⁺ P-SSCs express CCR5 (92%) and CCR3 (90%) on the cell surface at levels comparable to CD45⁺ hematopoietic cells, while more differentiated Mx1⁺ cells (Mx1+SMA−) or αSMA⁺ cells (Mx1−SMA+) have much lower CCR5 expression (FIG. 4D). By contrast, a distinct Mx1⁺αSMA⁺ population (˜0.01%) was not observed and there was significant upregulation of CCR5 in Mx1⁺ progenitors (26% of Mx1⁺140a⁺) in the BM (FIG. 4E). In addition, there was no differential expression of CXCR4 (receptor for CXCL12; a potential BM-SSC migratory factor) in Mx1⁺αSMA⁺ P-SSCs, suggesting the presence of a CCR5-mediated regulatory mechanism in P-SSC migration (FIG. 4D, bottom).

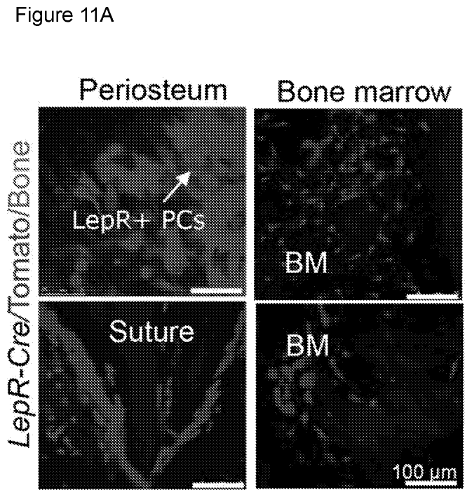

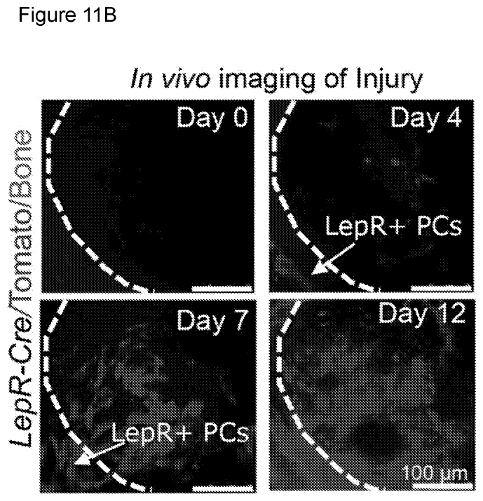

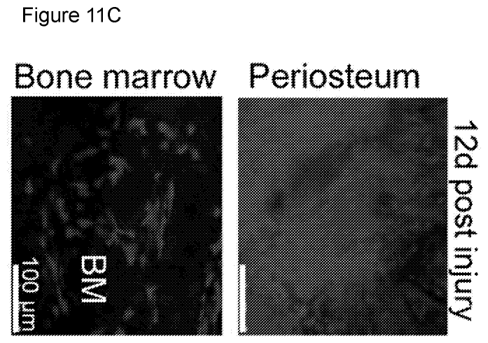

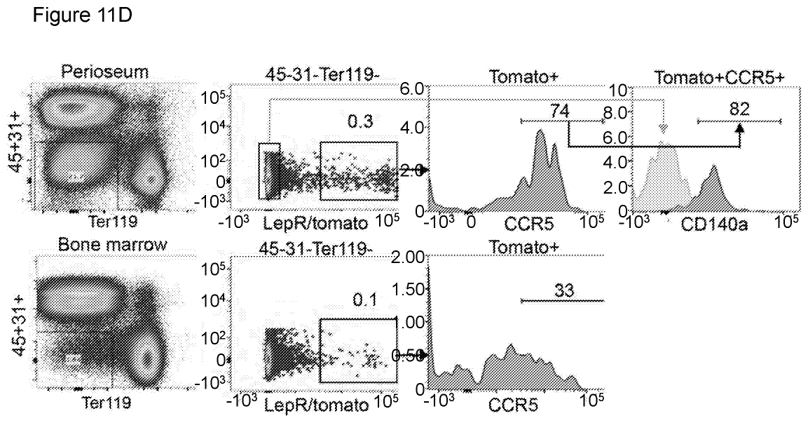

It may be possible that Mx1 does not label all SSCs in the BM and that Mx1⁻ BM-SSCs can express CCR3/5. Using LepR-Cre⁺Rosa26-Tomato⁺ reporter mice, generated to label postnatal BM-SSCs that are the major source of new osteoblasts and adipocytes in the BM (Zhou et al., 2014), LepR⁺ cells were tested in the BM for expression of CCR5 and their contribution to osteolineage cells in injury repair. In vivo imaging and FACS analysis of these mice revealed that the LepR⁺ cells are present in both the periosteum and the BM (FIG. 11A). When LepR⁺ cells were tracked in the fracture injury, it was consistently observed that periosteal LepR⁺ cells rapidly respond, proliferate, and contribute to injury healing, whereas no such migration and proliferation are observed in LepR⁺ cells in the BM (FIG. 11B & FIG. 11C). In addition, the majority of LepR⁺ periosteal cells express CCR5 (74%) with CD140a (82%), while LepR⁺ cells in the BM have no such expression (FIG. 11D), further demonstrating that CCR5 expression may specify endogenous P-SSCs that are functionally distinct from BM-SSCs.

A previous study reported that Prx1-GFP⁺ cells in the periosteum have progenitor characteristics and contribute to injury repair (Ouyang et al., 2013). Therefore, to exclude any possible contamination of CCR5⁺ hematopoietic cells in the Mx1⁺αSMA⁺ P-SSC fraction, FACS analysis of periosteal cells from Prx1-CreER-GFP mice was performed and it was confirmed that Prx1-GFP⁺ P-SSCs (CD45⁻CD31⁻TER119⁻CD140a⁺GFP⁺) have high expression of CCR5 (86%) on the cell surface (FIG. 4F). Intravital imaging of Mx1/Tomato/Prx1CreER-GFP mice without tamoxifen induction revealed that nearly all Prx1-GFP⁺ periosteal cells overlap with Mx1⁺ P-SSCs (Tomato⁺) in vivo (FIG. 4G), supporting that Mx1⁺ P-SSCs with Prx1-GFP expression have specific CCR5 expression that may regulate early P-SSC migration toward injury sites in vivo.

### Example 6

### CCL5 Induces the In Vivo Migration of P-SSCs

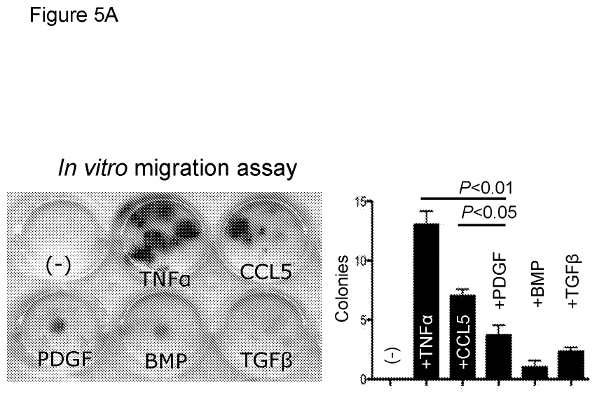

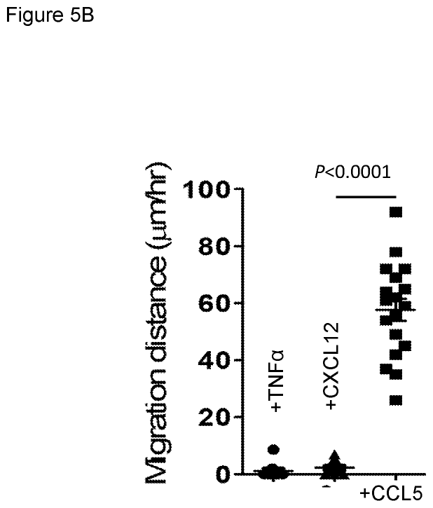

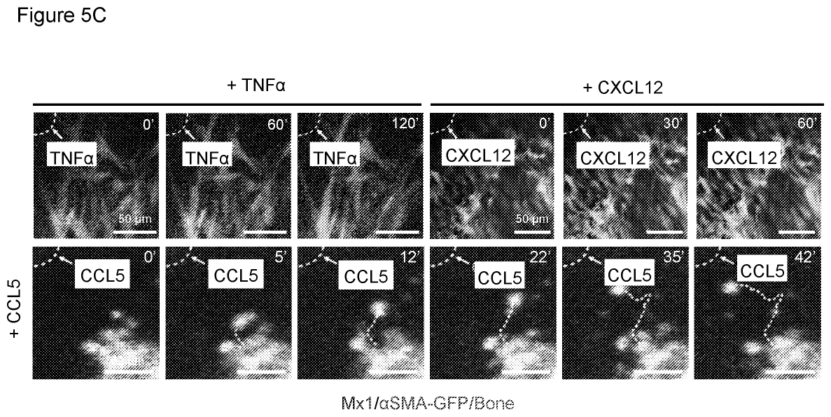

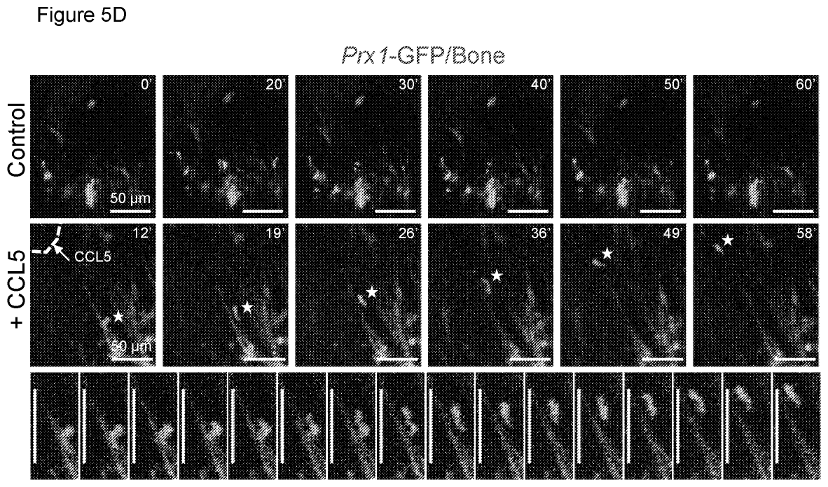

In vitro SSC migration followed by four-day colony formation assays with various bone growth factors revealed that tumor necrosis factor alpha (TNFα) and CCL5 (a common ligand for CCR3 and CCR5) (Blanpain et al., 2001) significantly enhance the migration capability of Mx1+P-SSCs (FIG. 5A). To test if CCL5 induces the migration of P-SSCs toward fracture sites in vivo, in vivo imaging of Mx1⁺αSMA⁺ P-SSCs was performed one hour after the administration of CCL5 (2 μL, 10 ng/μL in Matrigel) at a fracture injury site. Visualization of in vivo migration of Mx1⁺αSMA⁺ P-SSCs was possible by administering CCL5 locally (FIGS. 5B & FIG. 5C bottom), whereas in certain aspects various doses of TNFα or CXCL12 (10-50 ng in Matrigel) did not induce their migration in vivo (FIG. 5B & FIG. C top). CCL5 induces the directional migration of adjacent Mx1⁺αSMA⁺ P-SSCs toward the injury site while many distal P-SSCs remain stationary, with little movement. The total distance (i.e. multidimensional movement) of individual migrating cells (˜20 cells/group) was assessed for 2 hours (1 minute/frame), and the average migration speed under CCL5 administration was 57 μm/h, while there was no distinct movement with TNFα and CXCL12 treatment in this particular experiment (FIG. 5B). CCL5 induces the directional migration of Prx1-GFP⁺ P-SSCs toward the injury site with a similar migration speed (FIG. 5C), supporting that these CCL5-mediated migrating cells are the non-hematopoietic Mx1⁺αSMA⁺ P-SSC fraction. Despite a wide variation in the migration of each Mx1⁺αSMA⁺ P-SSCs due to a potential difference in CCL5 concentration at different locations at the time of imaging, the data indicate that P-SSCs are indeed dynamic and migrate upon CCL5 activation.

### Example 7

### CCL5 is Useful for Bone Healing, and Local Provision of CCL5 Accelerates Healing of Aged-Bone Defects with Increased P-SSC Recruitment

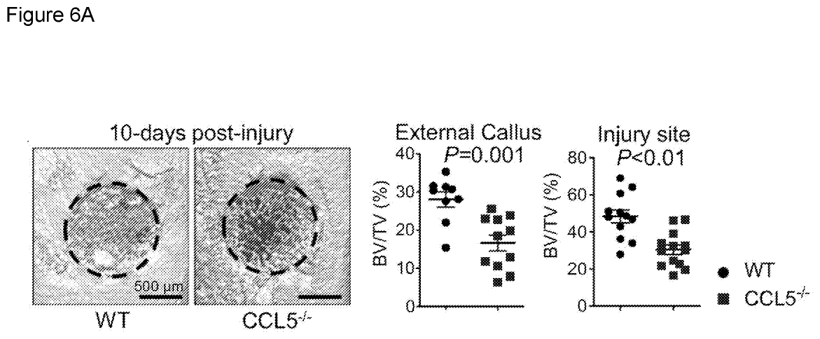

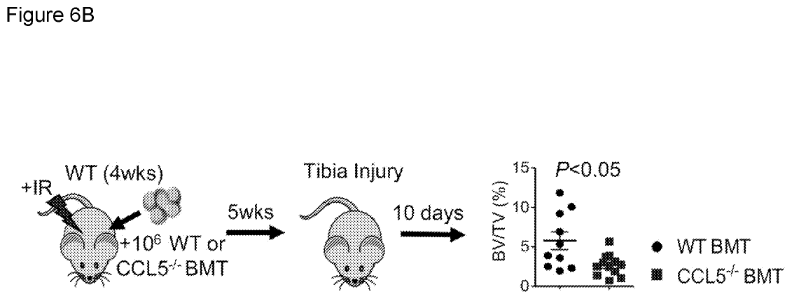

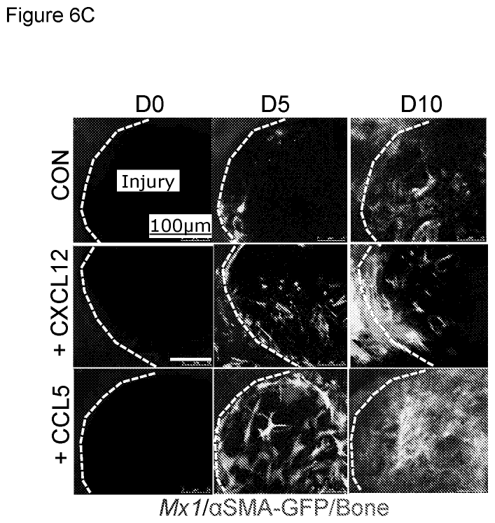

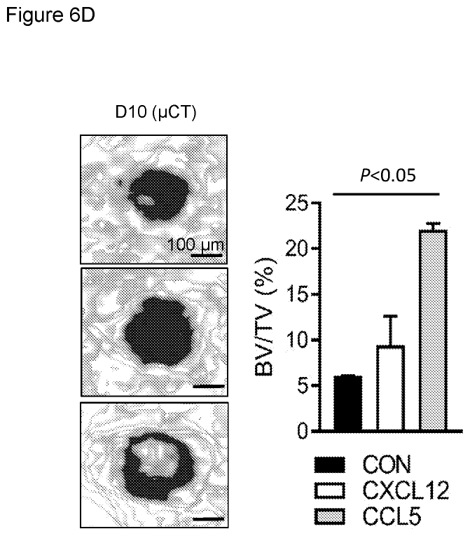

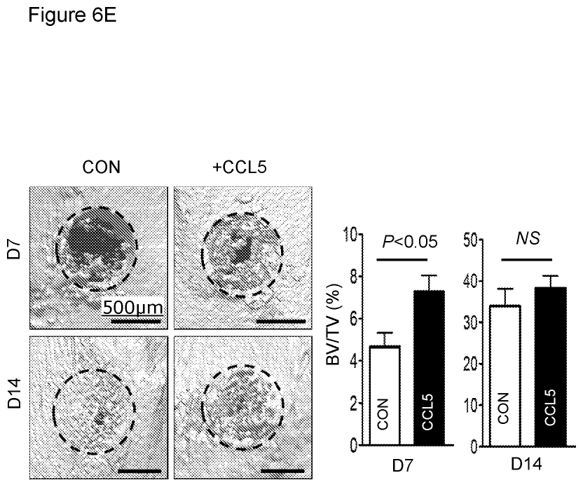

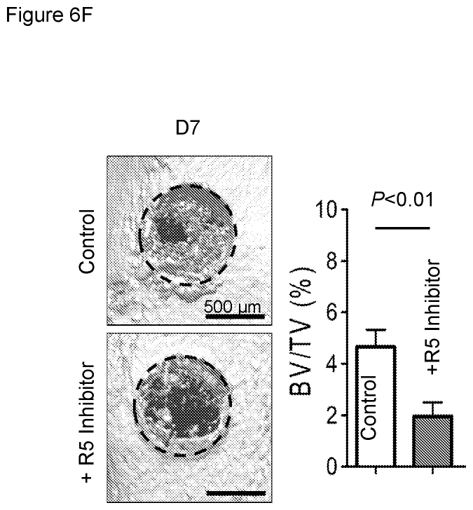

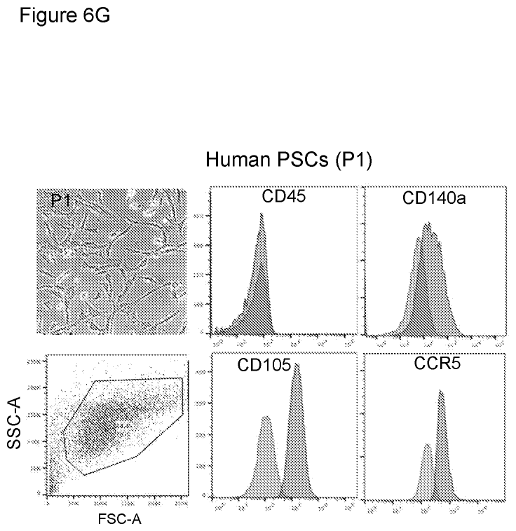

The development of novel therapeutic avenues to accelerate early bone healing will eventually benefit patients with devastating skeletal injuries. Given the CCL5-mediated migration of Mx1⁺αSMA⁺ P-SSCs and their recruitment toward injury sites, the in vivo contribution of CCL5 to the repair of fractured bones was assessed in an animal model. To test the physiologic importance of CCL5 in P-SSC migration and in injury repair, the bone healing of Ccl5-deficient mice was compared to that of WT litter mates. While there were no noticeable differences in bone parameters (BV/TV, travecular numbers and trabecular thickness) of 3-month-old Ccl5-deficient mice (data not shown), external callus mineralization (External BV/TV) and new bone formation (BV/TV) at defect sites 10 days after injury were significantly reduced (˜35%, p<0.01) in Ccl5-deficient mice compared to WT controls (FIG. 6A). Next, to test whether immune cells are the source of CCL5 for P-SSC migration and bone healing, WT mice were irradiated (9.5 Gy) and transplanted with Ccl5-deficient BM (10⁶ cells/mouse, Ccl5^{-/-} BMT) or WT BM as a control (WT-BMT). Five weeks after transplantation, a tibial drill-hole defect was induced and assessed bone healing 10 days after injury. A partial but significant delay in bone healing was observed in Ccl5^{-/-} BMT mice compared to WT-BMT mice (FIG. 6B). Consistent with previous reports showing CCL5 expression in multiple cell types upon injury (Aldinucci and Colombatti, 2014; Kovacic et al., 2010), these results imply that immune cells and other tissue cells express CCL5 at the injury site and contribute to P-SSC migration and bone healing.

Bone injuries were introduced to mouse calvaria of Mx1/Tomato/αSMA-GFP mice. Local treatment with CCL5 (+CCL5) or CXCL12 (+CXCL12) mixed with Matrigel (2 μL at 10 ng/μL) or Matrigel alone (CON) was given at the site of mechanical injury on days 0, 2, and 4. Treatment with CCL5 induced rapid recruitment of Mx1⁺αSMA⁺ P-SSCs at day 5 and substantially increased their number at day 10 with accelerated bone mineralization (FIG. 6C & FIG. 6D). Similarly, tibial injuries treated with CCL5 significantly increased early bone mineralization (FIG. 6E, D7), whereas treatment with a pharmacological CCR5 inhibitor (maraviroc) delayed bone healing (FIG. 6F). To confirm the findings, single-cell driven colonies from human periosteal tissues were cultured for 7 days. Cells were then analyzed for the expression of human SSC markers and CCR5. It was found that periosteal cells from both pooled culture and single-cell colonies express CCR5 and SSC markers (FIG. 6G), and that CCL5 treatment significantly induces human periosteal cell migration in vitro (FIG. 6H). These data indicate that local treatment with CCL5 enhances the recruitment of both mouse and human P-SSCs and may contribute to the recovery of fractured bones.

### Example 8

### The Deletion and Inhibition of CCR5 Delay P-SSC Migration and Bone Repair In Vivo

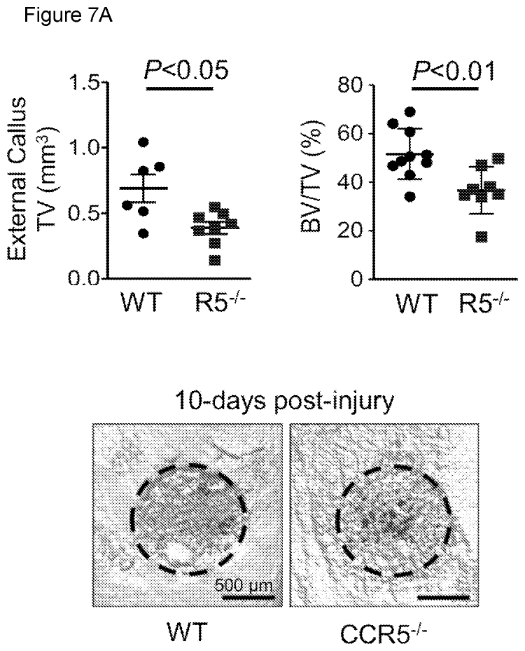

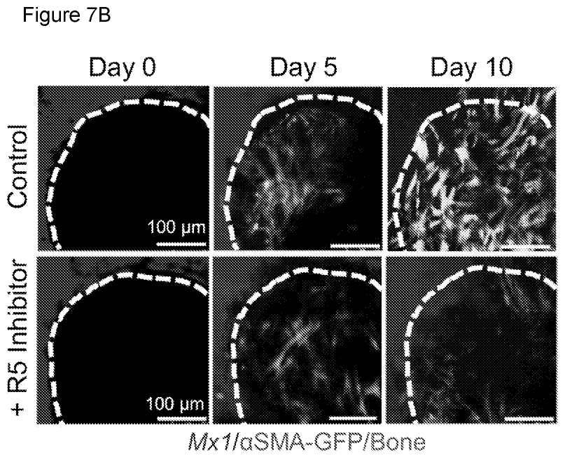

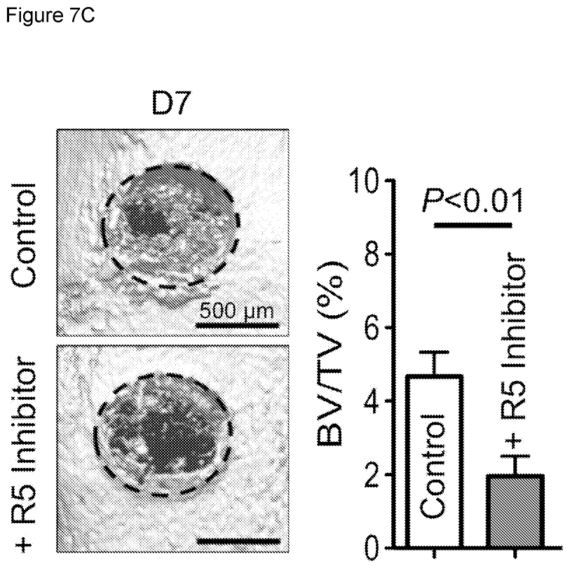

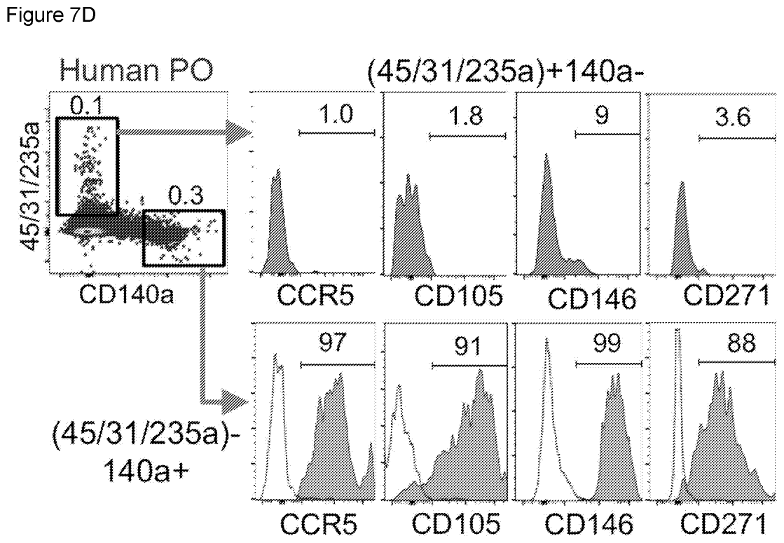

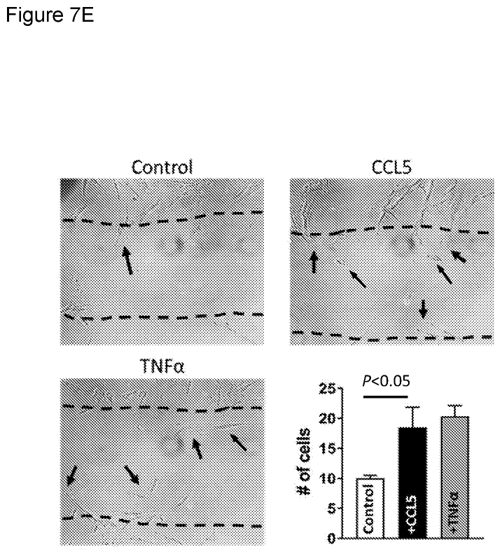

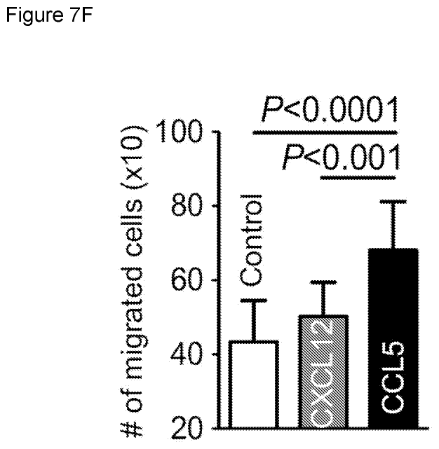

During live imaging of Mx1/Tom/SMA reporter mice treated with a pharmacological CCR5 inhibitor (FIG. 6F) a significant increase was noticed in the number and severity of periosteal resorption areas, whereas treatment with CCL5 lead to significantly greater recruitment of periosteal skeletal stem cells (PSSCs) and bone healing. To evaluate the function of CCR5 in bone healing, a tibial drill-hole defect (˜1 mm) model was used to assess bone healing at 10 days post injury in Ccr5-deficient mice. Ccr5-deficient mice displayed a significant reduction in external callus formation (TV) and bone healing (BV/TV) of defect sites compared to WT littermate controls (FIG. 7A). To further evaluate the specific function of CCR5 in Mx1⁺αSMA⁺ P-SSCs migration during early bone healing, calvarial injuries were induced in Mx1/Tomato/αSMA-GFP mice (2 months after pIpC and WT-BMT) and treated with a pharmacological CCR5 inhibitor (Maravoric) every other day for 10 days. Inhibition of CCR5 substantially reduced the recruitment of Mx1⁺αSMA⁺ P-SSCs 5 and 10 days post injury (FIG. 7B). Furthermore, a significant reduction of bone healing (BV/TV) was observed at 7 days after injury (FIG. 7C). These data indicate that CCL5-CCR5 signaling is an important component of P-SSC migration in response to injury. To confirm the translational implications, primary cells isolated from collagenase-digested human periosteal tissue were analyzed for the expression of human SSC markers and CCR5. It was found that CD45⁻CD31⁻CD235a⁻CD140a⁺ human periosteal cells expressed CCR5 as well as previously defined human SSC markers such as CD105, CD146, and CD271 (FIG. 7D). Consistent with FIG. 6C, treatment with CCL5, but not with CXCL12, significantly induced human periosteal cell migration in vitro (FIG. 7E) and in a transwell migration assay (FIG. 7F). These data indicate that local treatment with CCL5 enhances the recruitment of both mouse and human P-SSCs and may contribute to the recovery of fractured bones.

### Example 9

### In Vivo Imaging of Osteoclastic Response to Ccl5 or Inhibition of Ccr5

The assessment of the in vivo contribution of CCL5 or CCR5 inhibitor (maraviroc) treatments on osteoclast function in relation to bone healing was performed. An animal model was developed in which it is possible to track the osteoclast response to bone injury. CathepsinK (Ctsk) is a known marker of post-natal osteoclasts. Developmentally both mesenchymal and hematopoietic cell linages are Tom+ in CtskCre/Tom reporter mice, which is evident by the imbedding of Tom+ cells within the bone and abundance of Tom+ cells within the bone marrow. To achieve selective labeling of hematopoietic linage osteoclast, a bone marrow transplant into wild type (WT) recipient mice is necessary. Seven-week-old WT mice were lethally irradiated with 9.50 Gy and the following day 10 million CtskCre/Tom bone marrow cells were transplanted (CtskCre-BM). The bone marrow cells were allowed to repopulate for at least 6 weeks prior to experimentation. To test if stimulation or inhibition of CCR5 prevents or induces osteoclast differentiation and/or migration in response to an injury, in vivo intravital imaging of Ctsk⁺ cell response to a calvarial injury was performed.

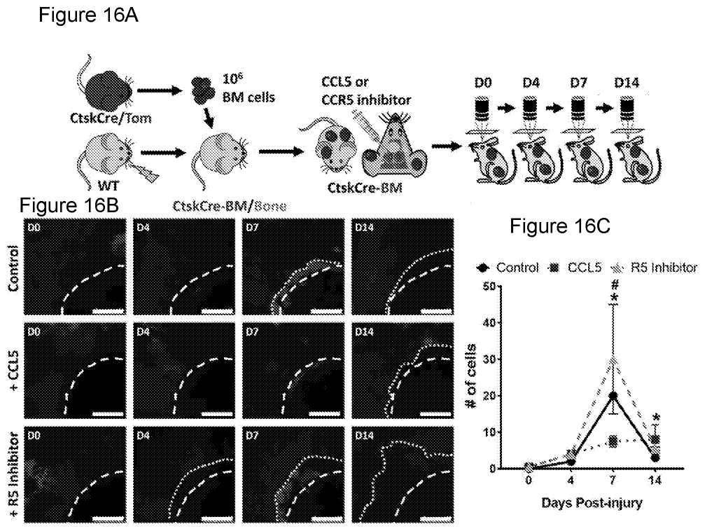

An injury was induced with a 27-gauge needle and mice received one of 3 treatments at the injury sight: 1) control (2 uL Matrigel) Day 0, 2, and 4; 2) CCL5 (2 uL, 10 ng/uL in Matrigel) Day 0, 2, and 4; or 3) a CCR5 inhibitor (10 uL, 4.9 mM Maraviroc in Matrigel) on Day 0, 2, 4, 7, and 10 post-injury (FIG. 16A). In vivo imaging occurred on the day of injury as well as 4, 7, and 14 days post injury. Under control conditions no Ctsk⁺ cells were present at the injury sight the day of injury. By Day 4, Ctsk⁺ cells were present, and a 10-fold increase in cell number occurring 7 days post injury (p<0.01). Fourteen days post-injury the number of Ctsk⁺ cells were comparable to day 4 post injury. Treatment of the injury sight with CCL5 significantly reduced the number of Ctsk cells present at the injury on Day 7. Interestingly, ˜50% more Ctsk cells were present day 14 with CCL5 treatment compared to the control (p<0.05), suggesting CCL5 lessens osteoclast response to injury. Treatment with the CCR5 inhibitor increased the number of Ctsk cells 7 days post injury. In addition to the number of Ctsk⁺ cells, osteoclastic resorption within the injury sight was apparent 4 days after injury and continued to expand with CCR5 inhibitor treatment compared to the control. Substantial resorption was not observed with CCL5 treatment until 14 days post injury, whereas resorption was present 7 days after injury in the control (FIG. 16B & FIG. 16C). Together these data support the hypothesis that CCL5 (or activation of CCR5) prevents osteoclast migration/differentiation in response to injury and inhibition of CCR5 stimulates osteoclast migration/differentiation and function.

### Example 10

### Injury Repair in CCL5^{-/-} Mice

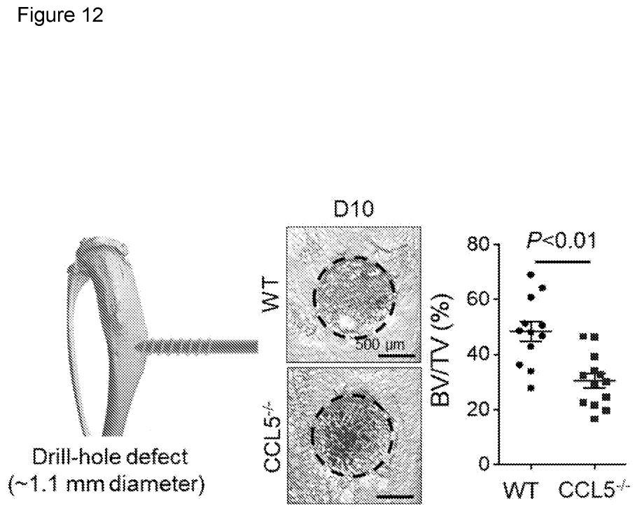

Based off findings that CCL5 treatment to bone injuries significantly increased the number of P-SSCs recruited to the injury sites and accelerates bone healing, the inventors next sought to determine if loss of CCL5 has deleterious effects on bone injury repair (see FIG. 12). Using age and sex-matched WT or 2-4-month-old CCL5^{-/-} mice, proximal tibia injuries were made on the anterior side of the proximal tibia using a 1.1 mm microdrill. Injured tibiae were collected ten-days post-injury and uCT imaging was performed. Injury repair was determined by the bone volume (BV) present in the total volume (TV) of the injury sites, and relative bone formation (BV/TV) was used to compare across groups to account for potential variability of the injury size. Loss of CCL5 resulted in an ˜35% reduction in early bone mineralization (WT: 48.5±3.5 vs. CCL5^{-/-}: 30.5±2.6, p<0.01). These data indicate that loss of CCL5 negatively impacts early bone healing and therefore plays an integral part in bone healing.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

SEQUENCES

SEQ ID NO: 1:

| | |
|---|---|
| 1 | gctgcagagg attcctgcag aggatcaaga cagcacgtgg acctcgcaca gcctctccca |
| 61 | caggtaccat gaaggtctcc gcggcagccc tcgctgtcat cctcattgct actgccctct |
| 121 | gcgctcctgc atctgcctcc ccatattcct cggacaccac accctgctgc tttgcctaca |
| 181 | ttgcccgccc actgccccgt gcccacatca aggagtattt ctacaccagt ggcaagtgct |
| 241 | ccaacccagc agtcgtccac aggtcaagga tgccaaagag agagggacag caagtctggc |
| 301 | aggatttcct gtatgactcc cggctgaaca agggcaagct ttgtcacccg aaagaaccgc |
| 361 | caagtgtgtg ccaacccaga gaagaaatgg gttcgggagt acatcaactc tttggagatg |
| 421 | agctaggatg gagagtcctt gaacctgaac ttacacaaat ttgcctgttt ctgcttgctc |
| 481 | ttgtcctagc ttgggaggct tcccctcact atcctacccc acccgctcct tgaagggccc |
| 541 | agattctacc acacagcagc agttacaaaa accttcccca ggctggacgt ggtggctcac |
| 601 | gcctgtaatc ccagcacttt gggaggccaa ggtgggtgga tcacttgagg tcaggagttc |
| 661 | gagaccagcc tggccaacat gatgaaaccc catctctact aaaaatacaa aaaattagcc |
| 721 | gggcgtggta gcgggcgcct gtagtcccag ctactcggga ggctgaggca ggagaatggc |
| 781 | gtgaacccgg gaggcggagc ttgcagtgag ccgagatcgc gccactgcac tccagcctgg |
| 841 | gcgacagagc gagactccgt ctcaaaaaaa aaaaaaaaaa aaaaaataca aaaattagcc |
| 901 | gggcgtggtg gcccacgcct gtaatcccag ctactcggga ggctaaggca ggaaaattgt |
| 961 | ttgaacccag gaggtggagg ctgcagtgag ctgagattgt gccacttcac tccagcctgg |
| 1021 | gtgacaaagt gagactccgt cacaacaaca acaacaaaaa gcttccccaa ctaaagccta |
| 1081 | gaagagcttc tgaggcgctg ctttgtcaaa aggaagtctc taggttctga gctctggctt |
| 1141 | tgccttggct ttgccagggc tctgtgacca ggaaggaagt cagcatgcct ctagaggcaa |
| 1201 | ggaggggagg aacactgcac tcttaagctt ccgccgtctc aacccctcac aggagcttac |
| 1261 | tggcaaacat gaaaaatcgg cttaccatta aagttctcaa tgcaaccata aaaaaaaaa |

SEQ ID NO: 2:

| | |
|---|---|
| 1 | gctgcagagg attcctgcag aggatcaaga cagcacgtgg acctcgcaca gcctctccca |
| 61 | caggtaccat gaaggtctcc gcggcagccc tcgctgtcat cctcattgct actgccctct |
| 121 | gcgctcctgc atctgcctcc ccatattcct cggacaccac accctgctgc tttgcctaca |
| 181 | ttgcccgccc actgccccgt gcccacatca aggagtattt ctacaccagt ggcaagtgct |
| 241 | ccaacccagc agtcgtcttt gtcacccgaa agaaccgcca agtgtgtgcc aacccagaga |
| 301 | agaaatgggt tcgggagtac atcaactctt tggagatgag ctaggatgga gagtccttga |
| 361 | acctgaactt acacaaattt gcctgtttct gcttgctctt gtcctagctt gggaggcttc |
| 421 | ccctcactat cctaccccac ccgctccttg aagggcccag attctaccac acagcagcag |
| 481 | ttacaaaaac cttccccagg ctggacgtgg tggctcacgc ctgtaatccc agcactttgg |
| 541 | gaggccaagg tgggtggatc acttgaggtc aggagttcga gaccagcctg gccaacatga |
| 601 | tgaaacccca tctctactaa aaatacaaaa aattagccgg gcgtggtagc gggcgcctgt |
| 661 | agtcccagct actcgggagg ctgaggcagg agaatggcgt gaacccggga ggcggagctt |
| 721 | gcagtgagcc gagatcgcgc cactgcactc cagcctgggc gacagagcga gactccgtct |
| 781 | caaaaaaaaa aaaaaaaaaa aaaatacaaa aattagccgg gcgtggtggc ccacgcctgt |
| 841 | aatcccagct actcgggagg ctaaggcagg aaaattgttt gaacccagga ggtggaggct |
| 901 | gcagtgagct gagattgtgc cacttcactc cagcctgggt gacaaagtga gactccgtca |
| 961 | caacaacaac aacaaaaagc ttccccaact aaagcctaga agagcttctg aggcgctgct |
| 1021 | ttgtcaaaag gaagtctcta ggttctgagc tctggctttg ccttggcttt gccagggctc |
| 1081 | tgtgaccagg aaggaagtca gcatgcctct agaggcaagg aggggaggaa cactgcactc |
| 1141 | ttaagcttcc gccgtctcaa cccctcacag gagcttactg gcaaacatga aaaatcggct |
| 1201 | taccattaaa gttctcaatg caaccataaa aaaaaaa |

SEQ ID NO: 3

| | |
|---|---|
| 1 | mkvsaaalav iliatalcap asaspyssdt tpccfayiar plprahikey fytsgkcsnp |
| 61 | avvhrsrmpk regqqvwqdf lydsrlnkgk lchpkeppsv cqpreemgsg vhqlfgdelg |
| 121 | wrvlepeltq iclfllalvl aweasphypt ppap |

SEQ ID NO: 4

| | |
|---|---|
| 1 | mkvsaaalav iliatalcap asaspyssdt tpccfayiar plprahikey fytsgkcsnp |
| 61 | avvfvtrknr qvcanpekkw vreyinslem s |

SEQ ID NO: 5

| | |
|---|---|
| 1 | cagacgctcc ctcagcaagg acagcagagg accagctaag agggagagaa gcaactacag |
| 61 | accccccctg aaaacaaccc tcagacgcca catcccctga caagctgcca ggcaggttct |
| 121 | cttcctctca catactgacc cacggctcca ccctctctcc cctggaaagg acaccatgag |
| 181 | cactgaaagc atgatccggg acgtggagct ggccgaggag gcgctcccca agaagacagg |
| 241 | ggggccccag ggctccaggc ggtgcttgtt cctcagcctc ttctccttcc tgatcgtggc |
| 301 | aggcgccacc acgctcttct gcctgctgca ctttggagtg atcggccccc agagggaaga |
| 361 | gttccccagg gacctctctc taatcagccc tctggcccag gcagtcagat catcttctcg |
| 421 | aaccccgagt gacaagcctg tagcccatgt tgtagcaaac cctcaagctg aggggcagct |
| 481 | ccagtggctg aaccgccggg ccaatgccct cctggccaat ggcgtggagc tgagagataa |
| 541 | ccagctggtg gtgccatcag agggcctgta cctcatctac tcccaggtcc tcttcaaggg |
| 601 | ccaaggctgc ccctccaccc atgtgctcct cacccacacc atcagccgca tcgccgtctc |
| 661 | ctaccagacc aaggtcaacc tcctctctgc catcaagagc ccctgccaga gggagacccc |
| 721 | agagggggct gaggccaagc cctggtatga gcccatctat ctgggagggg tcttccagct |
| 781 | ggagaagggt gaccgactca gcgctgagat caatcggccc gactatctcg actttgccga |
| 841 | gtctgggcag gtctactttg ggatcattgc cctgtgagga ggacgaacat ccaaccttcc |
| 901 | caaacgcctc ccctgcccca atccctttat taccccctcc ttcagacacc ctcaacctct |
| 961 | tctggctcaa aaagagaatt gggggcttag ggtcggaacc caagcttaga actttaagca |
| 1021 | acaagaccac cacttcgaaa cctgggattc aggaatgtgt ggcctgcaca gtgaagtgct |
| 1081 | ggcaaccact aagaattcaa actggggcct ccagaactca ctggggccta cagctttgat |
| 1141 | ccctgacatc tggaatctgg agaccaggga gcctttggtt ctggccagaa tgctgcagga |
| 1201 | cttgagaaga cctcacctag aaattgacac aagtggacct taggccttcc tctctccaga |
| 1261 | tgtttccaga cttccttgag acacggagcc cagccctccc catggagcca gctccctcta |
| 1321 | tttatgtttg cacttgtgat tatttattat ttatttatta tttatttatt tacagatgaa |
| 1381 | tgtatttatt tgggagaccg gggtatcctg ggggacccaa tgtaggagct gccttggctc |
| 1441 | agacatgttt tccgtgaaaa cggagctgaa caataggctg ttcccatgta gccccctggc |
| 1501 | ctctgtgcct tcttttgatt atgtttttta aaatatttat ctgattaagt tgtctaaaca |
| 1561 | atgctgattt ggtgaccaac tgtcactcat tgctgagcct ctgctcccca ggggagttgt |
| 1621 | gtctgtaatc gccctactat tcagtggcga gaaataaagt ttgcttagaa aagaaaaaaa |
| 1681 | aaaaaa |

SEQ ID NO: 6

| | |
|---|---|
| 1 | cagacgctcc ctcagcaagg acagcagagg accagctaag agggagagaa gcaactacag |
| 61 | accccccctg aaaacaaccc tcagacgcca catcccctga caagctgcca ggcaggttct |
| 121 | cttcctctca catactgacc cacggctcca ccctctctcc cctggaaagg acaccatgag |
| 181 | cactgaaagc atgatccggg acgtggagct ggccgaggag gcgctcccca agaagacagg |
| 241 | ggggccccag ggctccaggc ggtgcttgtt cctcagcctc ttctccttcc tgatcgtggc |
| 301 | aggcgccacc acgctcttct gcctgctgca ctttggagtg atcggccccc agagggaaga |
| 361 | gttccccagg gacctctctc taatcagccc tctggcccag gcagtcagat catcttctcg |
| 421 | aaccccgagt gacaagcctg tagcccatgt tgtagcaaac cctcaagctg aggggcagct |
| 481 | ccagtggctg aaccgccggg ccaatgccct cctggccaat ggcgtggagc tgagagataa |
| 541 | ccagctggtg gtgccatcag agggcctgta cctcatctac tcccaggtcc tcttcaaggg |
| 601 | ccaaggctgc ccctccaccc atgtgctcct cacccacacc atcagccgca tcgccgtctc |
| 661 | ctaccagacc aaggtcaacc tcctctctgc catcaagagc ccctgccaga gggagacccc |
| 721 | agagggggct gaggccaagc cctggtatga gcccatctat ctgggagggg tcttccagct |
| 781 | ggagaagggt gaccgactca gcgctgagat caatcggccc gactatctcg actttgccga |
| 841 | gtctgggcag gtctactttg ggatcattgc cctgtgagga ggacgaacat ccaaccttcc |
| 901 | caaacgcctc ccctgcccca atccctttat taccccctcc ttcagacacc ctcaacctct |
| 961 | tctggctcaa aaagagaatt gggggcttag ggtcggaacc caagcttaga actttaagca |
| 1021 | acaagaccac cacttcgaaa cctgggattc aggaatgtgt ggcctgcaca gtgaagtgct |
| 1081 | ggcaaccact aagaattcaa actggggcct ccagaactca ctggggccta cagctttgat |
| 1141 | ccctgacatc tggaatctgg agaccaggga gcctttggtt ctggccagaa tgctgcagga |
| 1201 | cttgagaaga cctcacctag aaattgacac aagtggacct taggccttcc tctctccaga |
| 1261 | tgtttccaga cttccttgag acacggagcc cagccctccc catggagcca gctccctcta |
| 1321 | tttatgtttg cacttgtgat tatttattat ttatttatta tttatttatt tacagatgaa |
| 1381 | tgtatttatt tgggagaccg gggtatcctg ggggacccaa tgtaggagct gccttggctc |
| 1441 | agacatgttt tccgtgaaaa cggagctgaa caataggctg ttcccatgta gccccctggc |
| 1501 | ctctgtgcct tcttttgatt atgtttttta aaatatttat ctgattaagt tgtctaaaca |
| 1561 | atgctgattt ggtgaccaac tgtcactcat tgctgagcct ctgctcccca ggggagttgt |
| 1621 | gtctgtaatc gccctactat tcagtggcga gaaataaagt ttgcttagaa aagaaaaaaa |
| 1681 | aaaaaa |

SEQ ID NO: 7

| | |
|---|---|
| 1 | ccacaaaggg cacttggccc cagggctagg agagcgaggg gagagcacag ccacccgcct |
| 61 | cggcggcccg ggactcggct cgactcgccg gagaatgcgc ccgaggacga cggggcgcca |
| 121 | gagccgcggt gctttcaact ggcgagcgcg aatgggggtg cactggagta aggcagagtg |
| 181 | atgcgggggg gcaactcgcc tggcaccgag atcgccgccg tgcccttccc tggacccggc |
| 241 | gtcgcccagg atggctgccc cgagccatgg gccgcggcgg agctagcgcg gagcgcccga |
| 301 | ccctcgaccc ccgagtcccg gagccggccc cgcgcggggc cacgcgtccc tcgggcgctg |
| 361 | gttcctaagg aggacgacag caccagcttc tcctttctcc cttcccttcc ctgccccgca |
| 421 | ctcctccccc tgctcgctgt tgttgtgtgt cagcacttgg ctggggactt cttgaacttg |
| 481 | cagggagaat aacttgcgca ccccactttg cgccggtgcc tttgccccag cggagcctgc |
| 541 | ttcgccatct ccgagcccca ccgcccctcc actcctcggc cttgcccgac actgagacgc |
| 601 | tgttcccagc gtgaaaagag agactgcgcg gccggcaccc gggagaagga ggaggcaaag |
| 661 | aaaaggaacg gacattcggt ccttgcgcca ggtcctttga ccagagtttt tccatgtgga |
| 721 | cgctctttca atggacgtgt ccccgcgtgc ttcttagacg gactgcggtc tcctaaaggt |
| 781 | cgaccatggt ggccgggacc cgctgtcttc tagcgttgct gcttccccag gtcctcctgg |
| 841 | gcggcgcggc tggcctcgtt ccggagctgg gccgcaggaa gttcgcggcg gcgtcgtcgg |
| 901 | gccgcccctc atcccagccc tctgacgagg tcctgagcga gttcgagttg cggctgctca |
| 961 | gcatgttcgg cctgaaacag agacccaccc ccagcaggga cgccgtggtg cccccctaca |
| 1021 | tgctagacct gtatcgcagg cactcaggtc agccgggctc acccgcccca gaccaccggt |
| 1081 | tggagagggc agccagccga gccaacactg tgcgcagctt ccaccatgaa gaatctttgg |
| 1141 | aagaactacc agaaacgagt gggaaaacaa cccggagatt cttctttaat ttaagttcta |
| 1201 | tccccacgga ggagtttatc acctcagcag agcttcaggt tttccgagaa cagatgcaag |
| 1261 | atgctttagg aaacaatagc agtttccatc accgaattaa tatttatgaa atcataaaac |
| 1321 | ctgcaacagc caactcgaaa ttccccgtga ccagactttt ggacaccagg ttggtgaatc |
| 1381 | agaatgcaag caggtgggaa agttttgatg tcacccccgc tgtgatgcgg tggactgcac |
| 1441 | agggacacgc caaccatgga ttcgtggtgg aagtggccca cttggaggag aaacaaggtg |
| 1501 | tctccaagag acatgttagg ataagcaggt ctttgcacca agatgaacac agctggtcac |
| 1561 | agataaggcc attgctagta acttttggcc atgatggaaa agggcatcct ctccacaaaa |
| 1621 | gagaaaaacg tcaagccaaa cacaaacagc ggaaacgcct taagtccagc tgtaagagac |
| 1681 | accctttgta cgtggacttc agtgacgtgg ggtggaatga ctggattgtg gctcccccgg |
| 1741 | ggtatcacgc cttttactgc cacggagaat gcccttttcc tctggctgat catctgaact |
| 1801 | ccactaatca tgccattgtt cagacgttgg tcaactctgt taactctaag attcctaagg |
| 1861 | catgctgtgt cccgacagaa ctcagtgcta tctcgatgct gtaccttgac gagaatgaaa |
| 1921 | aggttgtatt aaagaactat caggacatgg ttgtggaggg ttgtgggtgt cgctagtaca |
| 1981 | gcaaaattaa atacataaat atatatatat atatatattt tagaaaaaag aaaaaaacaa |
| 2041 | acaaacaaaa aaaccccacc ccagttgaca ctttaatatt tcccaatgaa gactttattt |
| 2101 | atggaatgga atggaaaaaa aaacagctat tttgaaaata tatttatatc tacgaaaaga |
| 2161 | agttgggaaa acaaatattt taatcagaga attattcctt aaagatttaa aatgtattta |
| 2221 | gttgtacatt ttatatgggt tcaaccccag cacatgaagt ataatggtca gatttatttt |
| 2281 | gtatttattt actattataa ccacttttta ggaaaaaaat agctaatttg tatttatatg |
| 2341 | taatcaaaag aagtatcggg tttgtacata attttccaaa aattgtagtt gttttcagtt |
| 2401 | gtgtgtattt aagatgaaaa gtctacatgg aaggttactc tggcaaagtg cttagcacgt |
| 2461 | ttgctttttt gcagtgctac tgttgagttc acaagttcaa gtccagaaaa aaaaagtgga |
| 2521 | taatccactc tgctgacttt caagattatt atattattca attctcagga atgttgcaga |
| 2581 | gtgattgtcc aatccatgag aatttacatc cttattaggt ggaatatttg gataagaacc |
| 2641 | agacattgct gatctattat agaaactctc ctcctgcccc ttaatttaca gaaagaataa |
| 2701 | agcaggatcc atagaaataa ttaggaaaac gatgaacctg caggaaagtg aatgatggtt |
| 2761 | tgttgttctt ctttcctaaa ttagtgatcc cttcaaaggg gctgatctgg ccaaagtatt |
| 2821 | caataaaacg taagatttct tcattattga tattgtggtc atatatattt aaaattgata |
| 2881 | tctcgtggcc ctcatcaagg gttggaaatt tatttgtgtt ttacctttac ctcatctgag |
| 2941 | agctctttat tctccaaaga acccagtttt ctaacttttt gcccaacacg cagcaaaatt |
| 3001 | atgcacatcg tgttttctgc ccaccctctg ttctctgacc tatcagcttg cttttctttc |
| 3061 | caaggttgtg tgtttgaaca catttctcca aatgttaaac ctatttcaga taataaatat |
| 3121 | caaatctctg gcatttcatt ctataaagtc caacctgtaa gagaaaaaaa aaaaaaaaaa |
| 3181 | aaaaaaaaaa a |

SEQ ID NO: 8

| | |
|---|---|
| 1 | mvagtrclla lllpqvllgg aaglvpelgr rkfaaassgr pssqpsdevl sefelrllsm |
| 61 | fglkqrptps rdavvppyml dlyrrhsgqp gspapdhrle raasrantvr sfhheeslee |
| 121 | lpetsgkttr rfffnlssip teefitsael qvfreqmqda lgnnssfhhr iniyeiikpa |
| 181 | tanskfpvtr lldtrlvnqn asrwesfdvt pavmrwtaqg hanhgfvvev ahleekqgvs |
| 241 | krhvrisrsl hqdehswsqi rpllvtfghd gkghplhkre krqakhkqrk rlkssckrhp |
| 301 | lyvdfsdvgw ndwivappgy hafychgecp fpladhlnst nhaivqtlvn svnskipkac |
| 361 | cvptelsais mlyldenekv vlknyqdmvv egcgcr |

1. A method of inducing bone healing in an individual comprising administering at least one cytokine to the individual at a site in need thereof, wherein the cytokine is selected from the group consisting of CCL5, TNFα, BMP2, Wnt, and a combination thereof.

2. The method of claim 1, wherein administering of the cytokine comprises delivery directly to the site of the individual.

3. The method of claim 1, wherein administering comprises delivery of the cytokine with a gel and/or scaffold.

4. The method of claim 3, wherein the gel comprises matrigel, hydrogel, periosteum, hydroxyapatite, tricalcium phosphate, polystyrene, poly-1-lactic acid, polyglycolic acid, poly-dl-lactic-co-glycolic acid, collagen, proteoglycans, alginate-based substrates, chitosan, collagen-GAG, extracellular matrix, or a combination thereof.

5. The method of claim 1, wherein the administering comprises delivery of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng of cytokine to the site in need.

6. (canceled)

7. The method of claim 1, wherein the delivery is by injection.

8. The method of claim 1, wherein the site in need comprises a bone fracture, bone break, bone degeneration, bone irradiation, a site of osteoporosis, or a combination thereof.

9. The method of claim 1, wherein the administering to the individual comprises administration of at least one cytokine and at least one additional therapy
wherein the at least one cytokine and the at least one additional therapy are administered to the individual at the same time or at different times,
wherein when they are administered at the same time, the at least one cytokine and the at least one additional therapy are administered to the individual in the same composition or in different compositions,
optionally wherein the additional therapy comprises surgical repair, fixation of a device to set the site in need, placing the individual in a cast or sling, or a combination thereof.

10. (canceled)

11. (canceled)

12. (canceled)

13. The method of claim 1, wherein the site in need is monitored for bone regeneration.

14. The method of claim 1, wherein the administration to the individual is prophylactic.

15. The method of claim 1, wherein the individual has osteoporosis, osteopetrosis, or osteosclerosis.

16. A method of recruiting periosteal skeletal stem cells to a site in an individual in need thereof, comprising administering at least one cytokine to the individual at the site in need, wherein the cytokine is selected from the group consisting of CCL5, TNFα, BMP2, Wnt, and a combination thereof.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A method of reducing osteoclast migration to a site in need in an individual, comprising administering at least one cytokine to the individual at the site in need, wherein the cytokine is selected from the group consisting of CCL5, TNFα, BMP2, Wnt, and a combination thereof.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A method of inhibiting bone formation at a site in need in an individual, comprising administering to an individual at least one agent that inhibits the function of CCR5 TNFα, BMP2, Wnt or a combination thereof.

44. The method of claim 43, wherein the agent is maraviroc or an antibody.

45. (canceled)

46. (canceled)

47. (canceled)

48. (canceled)

49. (canceled)

50. The method of claim 43, wherein the site in need comprises a bone spur or ectopic bone formation site or bunion.

51. (canceled)

52. (canceled)

53. (canceled)

54. (canceled)

55. (canceled)

56. (canceled)

57. A composition for administering to a bone site, comprising
(a) a cytokine and/or agent, and
(b) a gel and/or scaffold,
wherein the cytokine and/or agent comprises a chemokine, an inflammatory cytokine, a bone morphogenetic protein, a Wnt protein, a CCR5 receptor antagonist, an antibody, or a combination thereof.

58. The composition of claim 57, wherein the cytokine and/or agent comprises CCL5, TNFα, BMP2, Wnt, maraviroc, an antibody that targets CCL5, an antibody that targets TNFα, an antibody that targets BMP2, an antibody that targets Wnt, an antibody that targets CCR5 or a combination thereof.

59. The composition of claim 57, wherein the gel and/or scaffold comprises matrigel, hydrogel, periosteum, hydroxyapatite, tricalcium phosphate, polystyrene, poly-1-lactic acid, polyglycolic acid, poly-dl-lactic-co-glycolic acid, collagen, proteoglycans, alginate-based substrates, chitosan, collagen-GAG, extracellular matrix, or a combination thereof.


SEQUENCE LISTING (8 sequences)

SEQ ID NO: 1 - DNA, 1319 bp, Homo sapiens
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtccacaggtcaaggatgccaaagagagagggacagcaagtctggcaggatttcctgtatgactcccggctgaacaagggcaagctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 2 - DNA, 1237 bp, Homo sapiens
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 3 - protein, 154 aa, Homo sapiens
MKVSAAALAVILIATALCAPASASPYSSDTTPCCFAYIARPLPRAHIKEYFYTSGKCSNPAVVHRSRMPKREGQQVWQDFLYDSRLNKGKLCHPKEPPSVCQPREEMGSGVHQLFGDELGWRVLEPELTQICLFLLALVLAWEASPHYPTPPAP

SEQ ID NO: 4 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 5 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 6 - DNA, 1686 bp, Homo sapiens; identical to SEQ ID NO: 5

SEQ ID NO: 7 - DNA, 3191 bp, Homo sapiens
ccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtccaacctgtaagagaaaaaaaaaaaaaaaaaaaaaaaaaaaa

SEQ ID NO: 8 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR